(19) United States
(12) Patent Application Publication
(10) Pub. No.: US 2026/0144875 A1
(43) Pub. Date: May 28, 2026

(54) COMPOSITIONS AND METHODS FOR CROSSLINKING STRUCTURAL PROTEINS

(71) Applicant: ADS Therapeutics LLC, Irvine, CA (US)

(72) Inventors: Jinsong Ni, Irvine, CA (US); Wenkui Ken Fang, Irvine, CA (US); Rong Yang, Irvine, CA (US); Van Dinh, Irvine, CA (US)

(21) Appl. No.: 19/121,609

(22) PCT Filed: Dec. 21, 2023
(86) PCT No.: PCT/US2023/085324

§ 371 Date: Apr. 16, 2025

Related U.S. Application Data
(60) Provisional application No. 63/434,487, filed on Dec. 22, 2022.

(51) Int. Cl. A61K 41/00; A61K 47/14; A61P 27/02

(52) U.S. Cl. CPC A61K 41/0057; A61K 47/14; A61P 27/02

(57) ABSTRACT

The present application provides compounds, compositions, and methods for facilitating photo-crosslinking of structural proteins. In one example, the structural proteins are collagen and are present in a biological tissue. In one example, a pharmaceutical composition is provided, comprising a compound of Formula (II): (Formula (II), wherein R6, R7, R8, and R9 are as described herein. In one example, the composition comprises an organic vehicle, such as a semifluorinated alkane vehicle, a medium chain triglyceride vehicle, or a combination thereof. Methods of using the compositions are also provided and include medical applications in which tissue crosslinking is known to be beneficial. In one example, the disclosure provides methods of corneal crosslinking using the compositions.

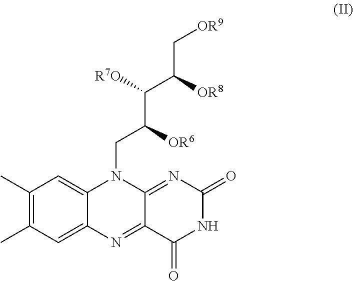

### CROSS-REFERENCE TO RELATED APPLICATIONS

This application claims the benefit of priority to U.S. Application No. 63/434,487, filed on Dec. 22, 2022, the contents of which are hereby incorporated by reference in its entirety.

### TECHNICAL FIELD

This disclosure relates to pharmaceutical compositions comprising riboflavin or riboflavin derivatives, and methods for crosslinking structural proteins such as, e.g., collagen or keratin, using the compositions.

### BACKGROUND

Various biological tissues or structural proteins found in biological tissues can be modified by crosslinking. For example, corneal tissue in mammals can be modified by photo-crosslinking the structural protein collagen in the corneal tissue. The crosslinking of collagen in the cornea can stabilize pathogenic or degenerative eye conditions, or correct vision defects. Riboflavin-5-phosphate has been used as a photosensitizer to facilitate in photo-crosslinking of collagen in corneal tissue.

### SUMMARY

This disclosure relates to compositions and methods for crosslinking structural proteins such as collagen. The structural protein can, in some embodiments, be present in a biological tissue. The tissue can, in some embodiments be ex vivo, or can be present in a subject such as a mammal.

This disclosure is based, at least in part, on a realization that certain riboflavin derivatives can be active in facilitating photo-crosslinking of structural proteins such as collagen. Without being bound by theory, it is believed that the derivatives of riboflavin described herein can act as (1) a photosensitizer to facilitate photo-crosslinking of structural proteins such as collagen on their own, and/or (2) can be converted to riboflavin within biological tissues and then act as a photosensitizer to facilitate photo-crosslinking of structural proteins. This disclosure is also based on the realization that the compositions and derivatives described herein can be useful at least in stable formulation of the photosensitizer, as well as in improved penetration and delivery of the compounds within biological tissues.

One nonlimiting example of a tissue that can benefit from crosslinking of, e.g., collagen is corneal tissue. Generally, from the outside inward, the cornea has a structure of 5 layers: (1) corneal epithelium, (2) Bowman's layer, (3) stroma, (4) Descemet's membrane, and (5) endothelium. In certain ocular conditions, such as, for example, keratoconus, the stroma of the cornea can thin or liquify, eventually leading to bulging of the cornea and subsequent vision changes. Corneal crosslinking (CXL) is a procedure that can be used to crosslink collagen fibrils within the stroma, thus increasing the mechanical strength of the stroma, altering its shape, and/or providing relief from various ocular symptoms and treating or halting the progression of ocular diseases such as keratoconus.

The Dresden Protocol, the original protocol developed for this procedure remains the standard CXL protocol in use, and generally involves de-epithelializing the cornea (removing at least a portion of the corneal epithelium), applying 0.1% riboflavin-5-phosphate in an aqueous solution, and applying UV radiation. See, e.g., Wollensak G, Spoerl E, Seiler T. Riboflavin/ultraviolet-a-induced collagen crosslinking for the treatment of keratoconus. Am J Ophthalmol 2003; 135:620-627. One reason for de-epithelializing the cornea for this procedure is because riboflavin and riboflavin-5-phosphate are hydrophilic molecules that do not penetrate the tight structure of the corneal epithelium well, thus reducing the amount of facilitator/photosensitizer that could reach the stroma for the crosslinking procedure. The procedure generally takes 30 minutes, and requires multiple reapplications of the riboflavin or riboflavin-5-phosphate due to its limitations, such as poor penetration into the corneal tissue and easy decomposition. Additionally, while the procedure can provide success in improving corneal conditions, it is not without side effects and complications. The de-epithelializing procedure required to allow the riboflavin to effectively penetrate into the cornea for crosslinking causes patients significant discomfort and leads to slow recovery time and opportunities for infection.

Various formulation attempts have been made to improve penetration of the riboflavin into the stroma without de-epithelialization, but many of these formulas contain irritating or even damaging components, such as ionic agents, that can irritate or damage tissues of the eye. Additionally, while these components may improve riboflavin penetration, they don't alter the highly hydrophilic nature of riboflavin itself. Therefore, the penetration efficiency of riboflavin is limited, even with the additional components. Mechanical methods for avoiding de-epithelialization have also been proposed but these often require specialized equipment and operating skills. Further, riboflavin and riboflavin-5-phosphate are highly susceptible to photodegradation. There remains a need for improved compounds, compositions, and methods for corneal crosslinking and crosslinking of other biological tissues.

This disclosure provides compositions comprising riboflavin or certain riboflavin derivatives that can be useful in crosslinking of structural proteins, and methods for crosslinking of structural proteins, including crosslinking of structural proteins in mammalian tissues. The compounds and compositions described herein provide improved photosensitizer/facilitator stability and delivery. For example, the compounds, compositions, and methods herein can, without the inclusion of irritants such as preservatives or ionic agents, in some embodiments, provide enhanced penetration of epithelial tissue, thus avoiding the need for de-epithelialization of the cornea in a corneal crosslinking procedure.

### In One Aspect, Described Herein is a Composition Comprising:

a compound of Formula I:

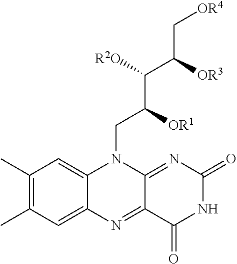

wherein:
   R1, R2, and R3 are each independently selected from hydrogen or —C(O)R⁵;
   R4 is selected from hydrogen, —C(O)R⁵, a phosphate, or a salt thereof; and
   R5 is a linear or branched or cyclic alkyl having 1 to 26 carbon atoms; and
a vehicle selected from a medium chain triglyceride (MCT), a semifluorinated alkane, or combinations thereof.

In this and other embodiments, Formula I can optionally have the following features. R1, R2, R3, and R4 can each be independently selected from hydrogen or —C(O)R⁵; R5 can be a linear or branched or cyclic alkyl having 1 to 26 carbon atoms; and at least one of R1, R2, R3, and R4 can be —C(O)R⁵, at least two of R1, R2, R3, and R4 can be —C(O)R⁵, or at least three of R1, R2, R3, and R4 can be —C(O) R⁵. R1, R2, R3, and R4 can each independently be —C(O)R⁵; and R5 can be a linear or branched or cyclic alkyl having 1 to 26 carbon atoms. R1, R2, R3, and R4 can each be independently selected from hydrogen or —C(O)R⁵; at least one of R1, R2, R3, and R4 can be —C(O)R⁵, at least two of R1, R2, R3, and R4 can be —C(O)R⁵, or at least three of R1, R2, R3, and R4 can be —C(O)R⁵; and R5 can be selected from a linear or branched alkyl having 1 to 3 or 15 carbon atoms. R1, R2, R3, and R4 can each independently be —C(O)R⁵, and R5 can be selected from a linear or branched alkyl having 1 to 3 or 15 carbon atoms. The compound of Formula I can, in some embodiments, be selected from riboflavin tetraacetate, riboflavin tetrapropionate, riboflavin tetrabutyrate, riboflavin tetrapalmitate, or combinations thereof.

In these and other embodiments, the compositions can optionally have the following features. The MCT can be a triglyceride of fatty acids, and the fatty acids are selected from the group consisting of hexanoic acid, octanoic acid, decanoic acid, dodecanoic acid, and combinations thereof. The semifluorinated alkane compound can have a formula of RFRH or a formula of RFRHRF; RF can be a perfluorinated hydrocarbon with 1 to 15 carbon atoms, and RH can be a non-fluorinated hydrocarbon with 1 to 15 carbon atoms. The semifluorinated alkane can be selected from perfluorobutylbutane (F4H4), perfluorobutylpentane (F4H5), perfluorobutylhexane (F4H6), perfluorohexylethane (F6H2), perfluorohexylbutane (F6H4), perfluorohexyloctane (F6H8), and perfluorohexyldecane (F6H10); preferably, perfluorohexylhexane (F6H6), and perfluorohexyloctane (F6H8). In some embodiments, the semifluorinated alkane can be perfluorohexyloctane (F6H8).

In these and other embodiments, the compound of Formula I can optionally be present in the composition(s) in an amount of from about 0.001% to about 1% (w/w), from about 0.01% to about 1% (w/w), from about 0.05% to about 0.5% (w/w), about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), or 1% (w/w).

In these and other embodiments, the MCT can optionally be present in the composition(s) in an amount of from about 0% to about 99.9% (w/w), from about 0.1% to about 99.9% (w/w), from about 0.1% to about 30% (w/w), from about 1% to about 25% (w/w), from about 1% to about 20% (w/w), from about 5% to about 20% (w/w), from about 5% to about 15% (w/w), from about 10% to about 99.9% (w/w), from about 20% to about 99.9% (w/w), from about 30% to about 99.9% (w/w), from about 40% to about 99.9% (w/w), from about 50% to about 99.9% (w/w), from about 60% to about 99.9% (w/w), from about 65% to about 99.9% (w/w), from about 70% to about 99.9% (w/w), from about 75% to about 99.9% (w/w), from about 80% to about 99.9% (w/w), from about 85% to about 99.9% (w/w), from about 90% to about 99.9% (w/w), from about 95% to about 99.9% (w/w), from about 98% to about 99.9% (w/w), from about 20% to about 80% (w/w), from about 20% to about 70% (w/w), from about 20% to about 60% (w/w), from about 20% to about 50% (w/w), from about 20% to about 40% (w/w), from about 20% to about 30% (w/w), from about 30% to about 80% (w/w), from about 40% to about 80% (w/w), from about 50% to about 80% (w/w), from about 60% to about 80% (w/w), from about 70% to about 80% (w/w), about 99.9% (w/w), about 99% (w/w), about 98% (w/w), about 97% (w/w), about 96% (w/w), about 95% (w/w), about 94% (w/w), about 93% (w/w), about 92% (w/w), about 91% (w/w), about 90% (w/w), about 85% (w/w), about 80% (w/w), about 75% (w/w), about 70% (w/w), about 65% (w/w), about 60% (w/w), about 55% (w/w), about 50% (w/w), about 45% (w/w), about 40% (w/w), about 35% (w/w), about 30% (w/w), about 25% (w/w), about 20% (w/w), about 15% (w/w), about 10% (w/w), about 9% (w/w), about 8% (w/w), about 7% (w/w), about 6% (w/w), about 5% (w/w), about 4% (w/w), about 3% (w/w), about 2% (w/w), about 1% (w/w), about 0.5% (w/w), or about 0.1% (w/w).

In these and other embodiments, the semifluorinated alkane can optionally be present in the composition(s) in an amount of from about 0% to about 99.9% (w/w), from about 0.1% to about 99.9% (w/w), from about 0.1% to about 30% (w/w), from about 1% to about 25% (w/w), from about 1% to about 20% (w/w), from about 5% to about 20% (w/w), from about 5% to about 15% (w/w), from about 10% to about 99.9% (w/w), from about 20% to about 99.9% (w/w), from about 30% to about 99.9% (w/w), from about 40% to about 99.9% (w/w), from about 50% to about 99.9% (w/w), from about 60% to about 99.9% (w/w), from about 65% to about 99.9% (w/w), from about 70% to about 99.9% (w/w), from about 75% to about 99.9% (w/w), from about 80% to about 99.9% (w/w), from about 85% to about 99.9% (w/w), from about 90% to about 99.9% (w/w), from about 95% to about 99.9% (w/w), from about 98% to about 99.9% (w/w), from about 20% to about 80% (w/w), from about 20% to about 70% (w/w), from about 20% to about 60% (w/w), from about 20% to about 50% (w/w), from about 20% to about 40% (w/w), from about 20% to about 30% (w/w), from about 30% to about 80% (w/w), from about 40% to about 80% (w/w), from about 50% to about 80% (w/w), from about 60% to about 80% (w/w), from about 70% to about 80% (w/w), about 99.9% (w/w), about 99% (w/w), about 98% (w/w), about 97% (w/w), about 96% (w/w), about 95% (w/w), about 94% (w/w), about 93% (w/w), about 92% (w/w), about 91% (w/w), about 90% (w/w), about 85% (w/w), about 80% (w/w), about 75% (w/w), about 70% (w/w), about 65% (w/w), about 60% (w/w), about 55% (w/w), about 50% (w/w), about 45% (w/w), about 40% (w/w), about 35% (w/w), about 30% (w/w), about 25% (w/w), about 20% (w/w), about 15% (w/w), about 10% (w/w), about 9% (w/w), about 8% (w/w), about 7% (w/w), about 6% (w/w), about 5% (w/w), about 4% (w/w), about 3% (w/w), about 2% (w/w), about 1% (w/w), about 0.5% (w/w), or about 0.1% (w/w).

In these and other embodiments, the composition(s) can have the following optional features. The MCT can be present in an amount of about 1% (w/w) and the SFA can be present in an amount of about 99% (w/w); the MCT can be present in an amount of about 2% (w/w) and the SFA can be present in an amount of about 98% (w/w); the MCT can be present in an amount of about 5% (w/w) and the SFA can be present in an amount of about 95% (w/w); the MCT can be present in an amount of about 10% (w/w) and the SFA can be present in an amount of about 90% (w/w); the MCT can be present in an amount of about 15% (w/w) and the SFA can be present in an amount of about 85% (w/w); the MCT can be present in an amount of about 20% (w/w) and the SFA can be present in an amount of about 80% (w/w); the MCT can be present in an amount of about 25% (w/w) and the SFA can be present in an amount of about 75% (w/w); the MCT can be present in an amount of about 30% (w/w) and the SFA can be present in an amount of about 70% (w/w); the MCT can be present in an amount of about 35% (w/w) and the SFA can be present in an amount of about 65% (w/w); the MCT can be present in an amount of about 40% (w/w) and the SFA can be present in an amount of about 60% (w/w); the MCT can be present in an amount of about 45% (w/w) and the SFA can be present in an amount of about 55% (w/w); the MCT can be present in an amount of about 50% (w/w) and the SFA can be present in an amount of about 50% (w/w); the MCT can be present in an amount of about 55% (w/w) and the SFA can be present in an amount of about 45% (w/w); the MCT can be present in an amount of about 60% (w/w) and the SFA can be present in an amount of about 40% (w/w); the MCT can be present in an amount of about 65% (w/w) and the SFA can be present in an amount of about 35% (w/w); the MCT can be present in an amount of about 70% (w/w) and the SFA can be present in an amount of about 30% (w/w); the MCT can be present in an amount of about 75% (w/w) and the SFA can be present in an amount of about 25% (w/w); the MCT can be present in an amount of about 80% (w/w) and the SFA can be present in an amount of about 20% (w/w); the MCT can be present in an amount of about 85% (w/w) and the SFA can be present in an amount of about 15% (w/w); the MCT can be present in an amount of about 90% (w/w) and the SFA can be present in an amount of about 10% (w/w); the MCT can be present in an amount of about 95% (w/w) and the SFA can be present in an amount of about 5% (w/w); the MCT can be present in an amount of about 98% (w/w) and the SFA can be present in an amount of about 2% (w/w); the MCT can be present in an amount of about 99% (w/w) and the SFA can be present in an amount of about 1% (w/w); MCT can be the only vehicle and the MCT can be present in an amount of about 99% (w/w); MCT can be the only vehicle and the MCT can be present in an amount of about 99.5% (w/w); MCT can be the only vehicle and the MCT can be present in an amount of about 99.6% (w/w); MCT can be the only vehicle and the MCT can be present in an amount of about 99.7% (w/w); MCT can be the only vehicle and the MCT can be present in an amount of about 99.8% (w/w); MCT can be the only vehicle and the MCT can be present in an amount of about 99.9% (w/w); SFA can be the only vehicle and the SFA can be present in an amount of about 99% (w/w); SFA can be the only vehicle and the SFA can be present in an amount of about 99.5% (w/w); SFA can be the only vehicle and the SFA can be present in an amount of about 99.6% (w/w); SFA can be the only vehicle and the SFA can be present in an amount of about 99.7% (w/w); SFA can be the only vehicle and the SFA can be present in an amount of about 99.8% (w/w), or wherein SFA can be the only vehicle and the SFA can be present in an amount of about 99.9% (w/w).

In these and other embodiments, the composition(s) can have the following optional features. The composition(s) can consist essentially of the compound of Formula I and the vehicle. The composition(s) can be an organic composition without aqueous components. The composition(s) can be a topical composition or a topical ocular composition, wherein the composition is a solution, a suspension, or an emulsion. The composition(s) can further comprise a viscosity enhancer. The composition(s) can be preservative-free or free of ocular irritants.

In another aspect, described herein is a method for crosslinking collagen in a cornea of a subject, comprising: administering to at least a portion of the cornea of the subject a composition as described above; and irradiating at least a portion of the cornea of the subject with UV radiation or blue light.

In another aspect, described herein is a method for crosslinking collagen in a tissue of a subject, comprising: administering to at least a portion of the cornea of the subject a composition as described above; and irradiating at least a portion of the cornea of the subject with UV radiation or blue light.

In another aspect, described herein is a method for crosslinking a structural protein, comprising: contacting the structural protein or a biological tissue containing the structural protein with a composition as described above; and irradiating the structural protein or the tissue of the subject with UV radiation or blue light.

In these and other embodiments of the methods described herein, the structural protein or, optionally, the collagen, can be present in a biological tissue or can be present in or on a body of a subject. The biological tissue can be ex vivo. The biological tissue can be in or on a body of a subject. Contacting the structural protein or contacting the tissue can comprise administering the composition to a tissue in a body of the subject. The tissue can be selected from a collagenous tissue, epidermal tissue, or keratinous tissue. The keratinous tissue is selected from hair or nails. The tissue can be selected from corneal tissue.

In these and other embodiments, the method(s) can have the following optional features. The subject can have a chemical burn or a disease selected from keratoconus, thin corneas, keratitis, infectious keratitis including bacterial or fungal keratitis, corneal ulcer, bullous keratopathy and other forms of corneal edema, keratolysis, an autoimmune disease, a cicatrizing disease such as Ocular Cicatricial Pemphigoid, Stevens-Johnson Syndrome, Lupus Erythematosus, Rheumatoid Arthritis, corneal ectasia including cornea ectasia appearing after laser-assisted in situ keratomileusis (LASIK) and photorefractive keratectomy (PRK), post-refractive ectasia, corneal degeneration, brittle cornea syndrome, Ehlers-Danlos Syndrome Type VI, corneal neovascularization, corneal melting including Corneal Melting in Boston Keratoprosthesis Type I, hyperopia, hyperopic astigmatism, myopia, astigmatism, myopic astigmatism, myopic regression, keratopathy, corneal astigmatism, unstable cornea, or pellucid marginal degeneration (PMD). The composition can be in the form of an eye drop and the method can further comprise applying the eye drop to an eye of the subject. The method can further comprise de-epithelializing at least a portion of the cornea. The crosslinking can be performed without de-epithelializing any portion of the cornea.

In another aspect, described herein is a method for crosslinking collagen in a tissue of a subject, comprising: administering to the tissue of the subject a compound of Formula II:

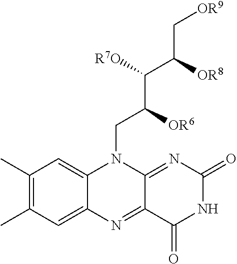

wherein:
   R6, R7, R8, and R9 are each independently selected from hydrogen or —C(O)R⁵;
   at least one of R6, R7, R8, and R9 is —C(O)R⁵; and
   R5 is selected from a linear or branched or cyclic alkyl having 1 to 26 carbon atoms; and
irradiating the tissue with UV radiation or blue light.

In another aspect, described herein is a method for crosslinking a structural protein, comprising: contacting the structural protein or a biological tissue containing the structural protein with a compound of Formula II:

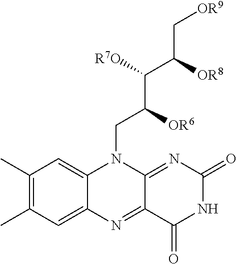

wherein:
   R6, R7, R8, and R9 are each independently selected from hydrogen or —C(O)R5;
   at least one of R6, R7, R8, and R9 is —C(O)R⁵; and
   R5 is selected from a linear or branched or cyclic alkyl having 1 to 26 carbon atoms; and
irradiating the structural protein or the tissue with UV radiation or blue light.

In these and other embodiments, Formula II can optionally have the following features. R1, R2, R3, and R4 can each independently be selected from hydrogen or —C(O)R⁵; R5 can be selected from a linear or branched or cyclic alkyl having 1 to 26 carbon atoms; and at least one of R1, R2, R3, and R4 can be —C(O)R⁵, at least two of R1, R2, R3, and R4 can be —C(O)R⁵, or at least three of R1, R2, R3, and R4 can be —C(O)R⁵. R1, R2, R3, and R4 can each be independently —C(O)R⁵; and R5 can be selected from a linear or branched or cyclic alkyl having 1 to 26 carbon atoms. R1, R2, R3, and R4 can each independently be selected from hydrogen or —C(O)R⁵; at least one of R1, R2, R3, and R4 can be —C(O)R⁵, at least two of R1, R2, R3, and R4 can be —C(O)R⁵, or at least three of R1, R2, R3, and R4 can be —C(O)R⁵; and R5 can be selected from a linear or branched alkyl having 1 to 3 or 15 carbon atoms. R1, R2, R3, and R4 can each independently be —C(O)R⁵, and R5 can be selected from a linear or branched alkyl having 1 to 3 or 15 carbon atoms. In some embodiments, the compound of Formula II can be selected from riboflavin tetraacetate, riboflavin tetrapropionate, riboflavin tetrabutyrate, riboflavin tetrapalmitate, or combinations thereof.

In these and other embodiments, the method can optionally have the following features. The structural protein can be selected from collagen, keratin, derivatives thereof, and combinations thereof. The structural protein can be present in a biological tissue or can be present in or on a body of a subject. The biological tissue can be ex vivo. The biological tissue can be in or on a body of a subject. Contacting the structural protein or contacting the tissue can comprise administering the compound of Formula II to a tissue in a body of the subject. The tissue can be selected from a collagenous tissue, epidermal tissue, or keratinous tissue. The keratinous tissue can be selected from hair or nails. Administering to the tissue a compound of Formula II can comprise administering to the tissue a composition comprising a compound of Formula II, and the composition can further comprise a vehicle. The vehicle can be selected from a medium chain triglyceride (MCT), a semifluorinated alkane, or combinations thereof. The MCT can be a triglyceride of fatty acids, and the fatty acids are selected from the group consisting of hexanoic acid, octanoic acid, decanoic acid, dodecanoic acid, and combinations thereof. The semifluorinated alkane compound can have a formula of RFRH or a formula of RFRHRF; RF can be a perfluorinated hydrocarbon with 1 to 15 carbon atoms, and RH can be a non-fluorinated hydrocarbon with 1 to 15 carbon atoms. The semifluorinated alkane can be selected from perfluorobutylbutane (F4H4), perfluorobutylpentane (F4H5), perfluorobutylhexane (F4H6), perfluorohexylethane (F6H2), perfluorohexylbutane (F6H4), perfluorohexyloctane (F6H8), and perfluorohexyldecane (F6H10); preferably, perfluorohexylhexane (F6H6), and perfluorohexyloctane (F6H8). The semifluorinated alkane can be perfluorohexyloctane (F6H8).

In these and other embodiments, the compound of Formula II can be present in the composition(s) in an amount of from about 0.001% to about 1% (w/w), from about 0.01% to about 1% (w/w), from about 0.05% to about 0.5% (w/w), about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), or 1% (w/w).

In these and other embodiments, the MCT can optionally be present in the composition(s) in an amount of from about 0% to about 99.9% (w/w), from about 0.1% to about 99.9% (w/w), from about 0.1% to about 30% (w/w), from about 1% to about 25% (w/w), from about 1% to about 20% (w/w), from about 5% to about 20% (w/w), from about 5% to about 15% (w/w), from about 10% to about 99.9% (w/w), from about 20% to about 99.9% (w/w), from about 30% to about 99.9% (w/w), from about 40% to about 99.9% (w/w), from about 50% to about 99.9% (w/w), from about 60% to about 99.9% (w/w), from about 65% to about 99.9% (w/w), from about 70% to about 99.9% (w/w), from about 75% to about 99.9% (w/w), from about 80% to about 99.9% (w/w), from about 85% to about 99.9% (w/w), from about 90% to about 99.9% (w/w), from about 95% to about 99.9% (w/w), from about 98% to about 99.9% (w/w), from about 20% to about 80% (w/w), from about 20% to about 70% (w/w), from about 20% to about 60% (w/w), from about 20% to about 50% (w/w), from about 20% to about 40% (w/w), from about 20% to about 30% (w/w), from about 30% to about 80% (w/w), from about 40% to about 80% (w/w), from about 50% to about 80% (w/w), from about 60% to about 80% (w/w), from about 70% to about 80% (w/w), about 99.9% (w/w), about 99% (w/w), about 98% (w/w), about 97% (w/w), about 96% (w/w), about 95% (w/w), about 94% (w/w), about 93% (w/w), about 92% (w/w), about 91% (w/w), about 90% (w/w), about 85% (w/w), about 80% (w/w), about 75% (w/w), about 70% (w/w), about 65% (w/w), about 60% (w/w), about 55% (w/w), about 50% (w/w), about 45% (w/w), about 40% (w/w), about 35% (w/w), about 30% (w/w), about 25% (w/w), about 20% (w/w), about 15% (w/w), about 10% (w/w), about 9% (w/w), about 8% (w/w), about 7% (w/w), about 6% (w/w), about 5% (w/w), about 4% (w/w), about 3% (w/w), about 2% (w/w), about 1% (w/w), about 0.5% (w/w), or about 0.1% (w/w).

In these and other embodiments, the semifluorinated alkane can optionally be present in the composition(s) in an amount of from about 0% to about 99.9% (w/w), from about 0.1% to about 99.9% (w/w), from about 0.1% to about 30% (w/w), from about 1% to about 25% (w/w), from about 1% to about 20% (w/w), from about 5% to about 20% (w/w), from about 5% to about 15% (w/w), from about 10% to about 99.9% (w/w), from about 20% to about 99.9% (w/w), from about 30% to about 99.9% (w/w), from about 40% to about 99.9% (w/w), from about 50% to about 99.9% (w/w), from about 60% to about 99.9% (w/w), from about 65% to about 99.9% (w/w), from about 70% to about 99.9% (w/w), from about 75% to about 99.9% (w/w), from about 80% to about 99.9% (w/w), from about 85% to about 99.9% (w/w), from about 90% to about 99.9% (w/w), from about 95% to about 99.9% (w/w), from about 98% to about 99.9% (w/w), from about 20% to about 80% (w/w), from about 20% to about 70% (w/w), from about 20% to about 60% (w/w), from about 20% to about 50% (w/w), from about 20% to about 40% (w/w), from about 20% to about 30% (w/w), from about 30% to about 80% (w/w), from about 40% to about 80% (w/w), from about 50% to about 80% (w/w), from about 60% to about 80% (w/w), from about 70% to about 80% (w/w), about 99.9% (w/w), about 99% (w/w), about 98% (w/w), about 97% (w/w), about 96% (w/w), about 95% (w/w), about 94% (w/w), about 93% (w/w), about 92% (w/w), about 91% (w/w), about 90% (w/w), about 85% (w/w), about 80% (w/w), about 75% (w/w), about 70% (w/w), about 65% (w/w), about 60% (w/w), about 55% (w/w), about 50% (w/w), about 45% (w/w), about 40% (w/w), about 35% (w/w), about 30% (w/w), about 25% (w/w), about 20% (w/w), about 15% (w/w), about 10% (w/w), about 9% (w/w), about 8% (w/w), about 7% (w/w), about 6% (w/w), about 5% (w/w), about 4% (w/w), about 3% (w/w), about 2% (w/w), about 1% (w/w), about 0.5% (w/w), or about 0.1% (w/w).

In these and other embodiments, the composition(s) can have the following optional features. The MCT can be present in an amount of about 1% (w/w) and the SFA can be present in an amount of about 99% (w/w); the MCT can be present in an amount of about 2% (w/w) and the SFA can be present in an amount of about 98% (w/w); the MCT can be present in an amount of about 5% (w/w) and the SFA can be present in an amount of about 95% (w/w); the MCT can be present in an amount of about 10% (w/w) and the SFA can be present in an amount of about 90% (w/w); the MCT can be present in an amount of about 15% (w/w) and the SFA can be present in an amount of about 85% (w/w); the MCT can be present in an amount of about 20% (w/w) and the SFA can be present in an amount of about 80% (w/w); the MCT can be present in an amount of about 25% (w/w) and the SFA can be present in an amount of about 75% (w/w); the MCT can be present in an amount of about 30% (w/w) and the SFA can be present in an amount of about 70% (w/w); the MCT can be present in an amount of about 35% (w/w) and the SFA can be present in an amount of about 65% (w/w); the MCT can be present in an amount of about 40% (w/w) and the SFA can be present in an amount of about 60% (w/w); the MCT can be present in an amount of about 45% (w/w) and the SFA can be present in an amount of about 55% (w/w); the MCT can be present in an amount of about 50% (w/w) and the SFA can be present in an amount of about 50% (w/w); the MCT can be present in an amount of about 55% (w/w) and the SFA can be present in an amount of about 45% (w/w); the MCT can be present in an amount of about 60% (w/w) and the SFA can be present in an amount of about 40% (w/w); the MCT can be present in an amount of about 65% (w/w) and the SFA can be present in an amount of about 35% (w/w); the MCT can be present in an amount of about 70% (w/w) and the SFA can be present in an amount of about 30% (w/w); the MCT can be present in an amount of about 75% (w/w) and the SFA can be present in an amount of about 25% (w/w); the MCT can be present in an amount of about 80% (w/w) and the SFA can be present in an amount of about 20% (w/w); the MCT can be present in an amount of about 85% (w/w) and the SFA can be present in an amount of about 15% (w/w); the MCT can be present in an amount of about 90% (w/w) and the SFA can be present in an amount of about 10% (w/w); the MCT can be present in an amount of about 95% (w/w) and the SFA can be present in an amount of about 5% (w/w); the MCT can be present in an amount of about 98% (w/w) and the SFA can be present in an amount of about 2% (w/w); the MCT can be present in an amount of about 99% (w/w) and the SFA can be present in an amount of about 1% (w/w); MCT can be the only vehicle and the MCT can be present in an amount of about 99% (w/w); MCT can be the only vehicle and the MCT can be present in an amount of about 99.5% (w/w); MCT can be the only vehicle and the MCT can be present in an amount of about 99.6% (w/w); MCT can be the only vehicle and the MCT can be present in an amount of about 99.7% (w/w); MCT can be the only vehicle and the MCT can be present in an amount of about 99.8% (w/w); MCT can be the only vehicle and the MCT can be present in an amount of about 99.9% (w/w); SFA can be the only vehicle and the SFA can be present in an amount of about 99% (w/w); SFA can be the only vehicle and the SFA can be present in an amount of about 99.5% (w/w); SFA can be the only vehicle and the SFA can be present in an amount of about 99.6% (w/w); SFA can be the only vehicle and the SFA can be present in an amount of about 99.7% (w/w); SFA can be the only vehicle and the SFA can be present in an amount of about 99.8% (w/w), or wherein SFA can be the only vehicle and the SFA can be present in an amount of about 99.9% (w/w).

In these and other embodiments of the method(s), the composition(s) can have the following optional features. The composition(s) can consist essentially of the compound of Formula II and the vehicle. The composition(s) can be an organic composition without aqueous components. The composition(s) can be a topical composition or a topical ocular composition. The composition(s) can be a solution, a suspension, or an emulsion. The composition(s) can further comprise a viscosity enhancer. The composition(s) can be preservative-free or free of ocular irritants.

In these and other embodiments, the method(s) can have the following optional features. The tissue can be a corneal tissue. The subject can have a chemical burn or a disease selected from keratoconus, thin corneas, keratitis, infectious keratitis including bacterial or fungal keratitis, corneal ulcer, bullous keratopathy and other forms of corneal edema, keratolysis, an autoimmune disease, a cicatrizing disease such as Ocular Cicatricial Pemphigoid, Stevens-Johnson Syndrome, Lupus Erythematosus, Rheumatoid Arthritis, corneal ectasia including cornea ectasia appearing after laser-assisted in situ keratomileusis (LASIK) and photorefractive keratectomy (PRK), post-refractive ectasia, corneal degeneration, brittle cornea syndrome, Ehlers-Danlos Syndrome Type VI, corneal neovascularization, corneal melting including Corneal Melting in Boston Keratoprosthesis Type I, hyperopia, hyperopic astigmatism, myopia, astigmatism, myopic astigmatism, myopic regression, keratopathy, corneal astigmatism, unstable cornea, or pellucid marginal degeneration (PMD). The composition can be in the form of an eye drop and the method can further comprise applying the eye drop to an eye of the subject. The method can further comprise de-epithelializing at least a portion of the cornea. The crosslinking can be performed without de-epithelializing any portion of the cornea.

In some embodiments, the compositions and methods provided herein can provide several advantages. First, in some embodiments of the compositions provided herein contain riboflavin derivatives with variable hydrophobicity, thus allowing tweaking by the skilled person for the optimal penetration, stability, and efficacy (as compared to that of riboflavin or riboflavin-5-phosphate) for the compositions' intended use.

Second, in some embodiments of the compositions and methods provided herein, the compounds can better penetrate a target tissue such as, for example, the corneal epithelium. This provides several additional advantages, including, in some embodiments, improved crosslinking within tissues because the compounds can reach the intended structural proteins for crosslinking. Further, the increased penetration can, in some embodiments, lead to using lower amounts or dosages of the compounds or compositions, or fewer applications, or shorter amounts of time for treating a tissue. Additionally, improved penetration can remove the need for de-epithelializing target tissues, such as the corneal epithelium, thus reducing pain and discomfort for patients, as well as reducing healing time and minimizing chances of infection.

Third, in some embodiments, the riboflavin derivative compounds described herein can exhibit greater stability than, e.g., riboflavin or riboflavin-5-phosphate, thus reducing the possibility of excess free radicals present in the tissue. In some embodiments, this improved stability can allow the use of compositions that are preservative-free or free of additional penetration enhancers or other irritants used in aqueous formulations to confer stability or enhance absorption through the corneal tissue, thus simplifying the compositions and reducing the possibility of irritation, such as eye irritation, or allergic reactions related to the inclusion of such additives. In some embodiments of the riboflavin derivative compounds described herein, the compounds can be less susceptible to photodegradation than riboflavin or riboflavin-5-phosphate, and yet still act as a photosensitizer for photo-crosslinking of structural proteins such as collagen. Without being bound by theory, it is believed that in some embodiments, the compounds can act as a photosensitizer either directly, or through activity similar to that of a prodrug in which the compounds enter the crosslinking environment, such as corneal tissue, and are then converted to ordinary riboflavin within the environment (for example, a mammalian eye). In either case, these embodiments can provide both the enhanced penetration and the desired crosslinking activity.

Fourth, in some embodiments described herein, the compounds can be formulated in a composition comprising an organic vehicle, such as, for example, a semifluorinated alkane (SFA) and/or a medium chain triglyceride (MCT). These formulations can, in some embodiments, allow increased stability of the compounds and therefore the formulations, the desired dosages, and/or a lack of impurities.

Other features and advantages of the present application will be apparent from the following detailed description and FIGURES, and from the claims.

### DETAILED DESCRIPTION

Provided herein are compositions which can facilitate photo-crosslinking of structural proteins such as collagen, and methods for crosslinking structural proteins, such as collagen and keratin, using compounds and compositions described further herein. In some embodiments, the structural protein can be in a tissue or in or on a body of a subject such as a mammal, e.g., a human.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

As used herein, the term “about” means “approximately” (e.g., plus or minus approximately 10% of the indicated value).

The term “alkyl,” as used herein, refers to saturated, monovalent hydrocarbon moieties having linear or branched moieties or combinations thereof and containing the specified (e.g., 1 to 26) carbon atoms.

The term “cycloalkyl,” as used herein, refers to a monovalent or divalent group of, e.g., 3 to 8 carbon atoms, derived from a saturated cyclic hydrocarbon. Cycloalkyl groups can be monocyclic or polycyclic.

The formula “H”, as used herein, represents a hydrogen atom.

The formula “O”, as used herein, represents an oxygen atom.

The formula “P”, as used herein, represents a phosphorus atom.

The term “compound” as used herein is meant to include all stereoisomers, geometric isomers, tautomers, and isotopes of the structures depicted, including racemic mixtures. Compounds herein identified by name or structure as one particular tautomeric form are intended to include other tautomeric forms unless otherwise specified.

As used herein, the term “contacting” refers to the bringing together of indicated moieties in an in vitro system or an in vivo system. For example, “contacting” the eye tissue with a compound of the invention includes the administration of a compound of the present invention to an individual or patient, such as a human, having the eye tissue in need of treatment, as well as, for example, introducing a compound of the invention into a sample containing a cellular or purified preparation containing the ocular tissue.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans. In some embodiment, a human subject may be of any age, for example, from 0 to 100 years old. As used herein, “pediatric subject” and “child” are used interchangeably and refer to subjects from 0 to 18 years old, for example, 1, 2, 5, 10, 12, or 14 years old.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

In some embodiments, provided herein are compositions comprising a compound that has the general Formula (I):

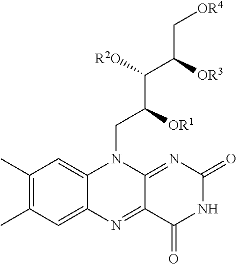

### In Formula (I)

R1, R2, and R3 are each independently selected from hydrogen or —C(O)R⁵;
R4 is selected from hydrogen, —C(O)R⁵, a phosphate, or a salt thereof; and
R5 is a linear or branched or cyclic alkyl having 1 to 26 carbon atoms (e.g., methyl, ethyl, propyl, butyl, sec-butyl, tert-butyl, and the like).

In some embodiments of Formula (I), R1, R2, R3, and R4 are each independently selected from hydrogen or —C(O)R⁵. In some embodiments of Formula (I), at least one of R1, R2, R3, and R4 is —C(O)R⁵, at least two of R1, R2, R3, and R4 are —C(O)R⁵, or at least three of R1, R2, R3, and R4 are —C(O)R⁵, or all of R1, R2, R3, and R4 are —C(O)R⁵.

In some embodiments, each of R1, R2, R3, and R4 can be the same groups or can have different groups.

In some embodiments of Formula (I), R1, R2, R3, and R4 are each independently selected from hydrogen or —C(O)R⁵; and at least one of R1, R2, R3, and R4 is —C(O)R⁵, at least two of R1, R2, R3, and R4 is —C(O)R⁵, or at least three of R1, R2, R3, and R4 is-C(O)R⁵. In some embodiments, R1, R2, R3, and R4 are each independently —C(O)R⁵.

In some embodiments of Formula (I), R1, R2, R3, and R4 are each independently selected from hydrogen or —C(O)R⁵; at least one of R1, R2, R3, and R4 is —C(O)R⁵, at least two of R1, R2, R3, and R4 are —C(O)R⁵, or at least three of R1, R2, R3, and R4 are —C(O)R⁵; and R5 can be a linear or branched alkyl having 1 or 3 carbon atoms. In some embodiments, R1, R2, R3, and R4 are each independently —C(O)R⁵, and R5 can be a linear or branched alkyl having 1 or 3 carbon atoms. In some embodiments, exemplary compounds of Formula (I) can include riboflavin, riboflavin-5-phosphate, riboflavin tetraacetate: (2R,3S,4S)-5-(7,8-dimethyl-2,4-dioxo-3,4-dihydrobenzo[g]pteridin-10(2H)-yl) pentane-1,2,3,4-tetrayl tetraacetate, riboflavin tetrapropionate: (2R,3S,4S)-5-(7,8-dimethyl-2,4-dioxo-3,4-dihydrobenzo[g]pteridin-10(2H)-yl) pentane-1,2,3,4-tetrayl tetrapropionate, riboflavin tetrabutyrate: (2R,3S,4S)-5-(7,8-dimethyl-2,4-dioxo-3,4-dihydrobenzo[g]pteridin-10(2H)-yl) pentane-1,2,3,4-tetrayl tetrabutyrate, riboflavin tetrapalmitate: (2R,3S,4S)-5-(7,8-dimethyl-2,4-dioxo-3,4-dihydrobenzo[g]pteridin-10(2H)-yl) pentane-1,2,3,4-tetrayl tetrapalmitate, or combinations thereof.

In some embodiments, the compound of Formula (I) can be present in the compositions in an amount of from about 0.001% to about 1% (w/w), 0.001% to about 0.95% (w/w), 0.001% to about 0.9% (w/w), 0.001% to about 0.85% (w/w), 0.001% to about 0.8% (w/w), 0.001% to about 0.75% (w/w), 0.001% to about 0.7% (w/w), 0.001% to about 0.65% (w/w), 0.001% to about 0.6% (w/w), 0.001% to about 0.55% (w/w), 0.001% to about 0.5% (w/w), 0.001% to about 0.45% (w/w), 0.001% to about 0.4% (w/w), 0.001% to about 0.35% (w/w), 0.001% to about 0.3% (w/w), 0.001% to about 0.25% (w/w), 0.001% to about 0.2% (w/w), 0.001% to about 0.15% (w/w), 0.001% to about 0.1% (w/w), 0.001% to about 0.09% (w/w), 0.001% to about 0.08% (w/w), 0.001% to about 0.07% (w/w), 0.001% to about 0.06% (w/w), 0.001% to about 0.05% (w/w), 0.001% to about 0.04% (w/w), 0.001% to about 0.03% (w/w), 0.001% to about 0.02% (w/w), 0.001% to about 0.01% (w/w), from about 0.01% to about 1%, 0.01% to about 0.95% (w/w), 0.01% to about 0.9% (w/w), 0.01% to about 0.85% (w/w), 0.01% to about 0.8% (w/w), 0.01% to about 0.75% (w/w), 0.01% to about 0.7% (w/w), 0.01% to about 0.65% (w/w), 0.01% to about 0.6% (w/w), 0.01% to about 0.55% (w/w), 0.01% to about 0.5% (w/w), 0.01% to about 0.45% (w/w), 0.01% to about 0.4% (w/w), 0.01% to about 0.35% (w/w), 0.01% to about 0.3% (w/w), 0.01% to about 0.25% (w/w), 0.01% to about 0.2% (w/w), 0.01% to about 0.15% (w/w), 0.01% to about 0.1% (w/w), 0.01% to about 0.09% (w/w), 0.01% to about 0.08% (w/w), 0.01% to about 0.07% (w/w), 0.01% to about 0.06% (w/w), 0.01% to about 0.05% (w/w), 0.01% to about 0.04% (w/w), 0.01% to about 0.03% (w/w), 0.01% to about 0.02% (w/w), from about 0.05% to about 0.5% (w/w), from about 0.05% to about 0.45% (w/w), from about 0.05% to about 0.4% (w/w), from about 0.05% to about 0.35% (w/w), from about 0.05% to about 0.3% (w/w), from about 0.05% to about 0.25% (w/w), from about 0.05% to about 0.2% (w/w), from about 0.05% to about 0.15% (w/w), from about 0.05% to about 0.1% (w/w), from about 0.05% to about 0.09% (w/w), from about 0.05% to about 0.08% (w/w), from about 0.05% to about 0.07% (w/w), from about 0.05% to about 0.06% (w/w), from about 0.06% to about 0.5% (w/w), from about 0.07% to about 0.5% (w/w), from about 0.08% to about 0.5% (w/w), from about 0.09% to about 0.5% (w/w), from about 0.1% to about 0.5% (w/w), from about 0.15% to about 0.5% (w/w), from about 0.2% to about 0.5% (w/w), from about 0.25% to about 0.5% (w/w), from about 0.3% to about 0.5% (w/w), from about 0.35% to about 0.5% (w/w), from about 0.4% to about 0.5% (w/w), from about 0.45% to about 0.5% (w/w), about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.15% (w/w), 0.2% (w/w), 0.25% (w/w), 0.3% (w/w), 0.35% (w/w), 0.4% (w/w), 0.45% (w/w), 0.5% (w/w), 0.55% (w/w), 0.6% (w/w), 0.65% (w/w), 0.7% (w/w), 0.75% (w/w), 0.8% (w/w), 0.85% (w/w), 0.9% (w/w), 0.95% (w/w), or 1% (w/w).

In some embodiments, provided herein are compositions comprising a compound that has the general Formula (II):

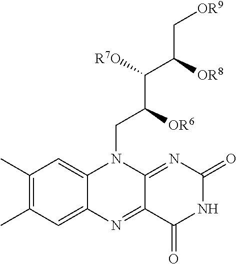

### In Formula (II)

R6, R7, R8, and R9 are each independently selected from hydrogen or —C(O)R⁵;
at least one of R6, R7, R8, and R9 is —C(O)R⁵; and
R5 is a linear or branched or cyclic alkyl having 1 to 26 carbon atoms.

In some embodiments of Formula (II), R6, R7, R8, and R9, at least one of R6, R7, R8, and R9 is —C(O)R⁵, at least two of R6, R7, R8, and R9 are —C(O)R⁵, or at least three of R6, R7, R8, and R9 are —C(O)R⁵, or all of R6, R7, R8, and R9 are —C(O)R⁵. In some embodiments, each of R6, R7, R8, and R9 can be the same groups or can have different groups.

In some embodiments of Formula (II), R6, R7, R8, and R9 are each independently selected from hydrogen or —C(O)R⁵; R5 can be selected from a linear or branched or cyclic alkyl having 1 to 26 carbon atoms; and at least two of R6, R7, R8, and R9 are —C(O)R⁵, or at least three of R6, R7, R8, and R9 are —C(O)R⁵.

In some embodiments of Formula (II), R6, R7, R8, and R9 are each independently —C(O)R⁵; and R5 can be selected from a linear or branched or cyclic alkyl having 1 to 26 carbon atoms.

In some embodiments of Formula (II), R6, R7, R8, and R9 are each independently selected from hydrogen or —C(O)R⁵; at least two of R6, R7, R8, and R9 are —C(O)R⁵, or at least three of R6, R7, R8, and R9 are —C(O)R⁵; and R5 can be selected from a linear or branched alkyl having 1 or 3 carbon atoms.

In some embodiments of Formula (II), R6, R7, R8, and R9 are each independently —C(O)R⁵, and R5 can be selected from a linear or branched alkyl having 1 or 3 carbon atoms. In some embodiments, exemplary compounds of Formula (II) can include selected from riboflavin tetraacetate, riboflavin tetrapropionate, riboflavin tetrabutyrate, riboflavin tetrapalmitate, or combinations thereof.

In some embodiments, the compound of Formula (II) can be present in the compositions in an amount of from about 0.001% to about 1% (w/w), 0.001% to about 0.95% (w/w), 0.001% to about 0.9% (w/w), 0.001% to about 0.85% (w/w), 0.001% to about 0.8% (w/w), 0.001% to about 0.75% (w/w), 0.001% to about 0.7% (w/w), 0.001% to about 0.65% (w/w), 0.001% to about 0.6% (w/w), 0.001% to about 0.55% (w/w), 0.001% to about 0.5% (w/w), 0.001% to about 0.45% (w/w), 0.001% to about 0.4% (w/w), 0.001% to about 0.35% (w/w), 0.001% to about 0.3% (w/w), 0.001% to about 0.25% (w/w), 0.001% to about 0.2% (w/w), 0.001% to about 0.15% (w/w), 0.001% to about 0.1% (w/w), 0.001% to about 0.09% (w/w), 0.001% to about 0.08% (w/w), 0.001% to about 0.07% (w/w), 0.001% to about 0.06% (w/w), 0.001% to about 0.05% (w/w), 0.001% to about 0.04% (w/w), 0.001% to about 0.03% (w/w), 0.001% to about 0.02% (w/w), 0.001% to about 0.01% (w/w), from about 0.01% to about 1%, 0.01% to about 0.95% (w/w), 0.01% to about 0.9% (w/w), 0.01% to about 0.85% (w/w), 0.01% to about 0.8% (w/w), 0.01% to about 0.75% (w/w), 0.01% to about 0.7% (w/w), 0.01% to about 0.65% (w/w), 0.01% to about 0.6% (w/w), 0.01% to about 0.55% (w/w), 0.01% to about 0.5% (w/w), 0.01% to about 0.45% (w/w), 0.01% to about 0.4% (w/w), 0.01% to about 0.35% (w/w), 0.01% to about 0.3% (w/w), 0.01% to about 0.25% (w/w), 0.01% to about 0.2% (w/w), 0.01% to about 0.15% (w/w), 0.01% to about 0.1% (w/w), 0.01% to about 0.09% (w/w), 0.01% to about 0.08% (w/w), 0.01% to about 0.07% (w/w), 0.01% to about 0.06% (w/w), 0.01% to about 0.05% (w/w), 0.01% to about 0.04% (w/w), 0.01% to about 0.03% (w/w), 0.01% to about 0.02% (w/w), from about 0.05% to about 0.5% (w/w), from about 0.05% to about 0.45% (w/w), from about 0.05% to about 0.4% (w/w), from about 0.05% to about 0.35% (w/w), from about 0.05% to about 0.3% (w/w), from about 0.05% to about 0.25% (w/w), from about 0.05% to about 0.2% (w/w), from about 0.05% to about 0.15% (w/w), from about 0.05% to about 0.1% (w/w), from about 0.05% to about 0.09% (w/w), from about 0.05% to about 0.08% (w/w), from about 0.05% to about 0.07% (w/w), from about 0.05% to about 0.06% (w/w), from about 0.06% to about 0.5% (w/w), from about 0.07% to about 0.5% (w/w), from about 0.08% to about 0.5% (w/w), from about 0.09% to about 0.5% (w/w), from about 0.1% to about 0.5% (w/w), from about 0.15% to about 0.5% (w/w), from about 0.2% to about 0.5% (w/w), from about 0.25% to about 0.5% (w/w), from about 0.3% to about 0.5% (w/w), from about 0.35% to about 0.5% (w/w), from about 0.4% to about 0.5% (w/w), from about 0.45% to about 0.5% (w/w), about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.15% (w/w), 0.2% (w/w), 0.25% (w/w), 0.3% (w/w), 0.35% (w/w), 0.4% (w/w), 0.45% (w/w), 0.5% (w/w), 0.55% (w/w), 0.6% (w/w), 0.65% (w/w), 0.7% (w/w), 0.75% (w/w), 0.8% (w/w), 0.85% (w/w), 0.9% (w/w), 0.95% (w/w), or 1% (w/w).

Compounds of Formula (I) or Formula (II) and their salts can be in the form of a solvate, which is included within the scope of the present invention. Such solvates include for example hydrates, alcoholates (e.g., ethanol solvate), and the like. Compounds according to the present invention may exist in different polymorphic forms. Although not explicitly indicated in the above formulas, such forms are intended to be included within the scope of the present disclosure.

In some embodiments, compositions comprising Formula (I), Formula (II), or combinations thereof can be pharmaceutical compositions. In some embodiments of the pharmaceutical compositions a compound disclosed herein (e.g., a compound of Formula (I)) can be present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as the use of other agents and the judgment of the treating physician. For example, in some embodiments, where the compositions described herein may be used to treat a disease in an eye of the patient, such as keratoconus, the treating physician may adjust the dosage and dosage regimen based on the thickness or condition of the patient's cornea.

In some embodiments, an effective amount (e.g., in the systemic formulation) of the compound (e.g., Formula (I) or a pharmaceutically acceptable salt thereof) can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound of Formula (I) is about 0.1 mg/kg, about 0.5 mg/kg, about 1 mg/kg, about 2 mg/kg, or about 5 mg/kg.

In some embodiments, the dosages can be administered on a daily basis (e.g., as a single dose or as two or more divided doses, e.g., once daily, two times daily, three times daily, or four times daily) or on a non-daily basis (e.g., every other day, every two days, every three days, once weekly, twice weekly, once every two weeks, once a month).

In some embodiments, the compositions can comprise a vehicle. The vehicle can act as a carrier or a solvent for one or more compounds of Formula (I), one or more compounds of Formula (II), or combinations thereof. In some embodiments, the composition can be in the form of a solution, a suspension, or an emulsion. In some embodiments, a composition provided herein can consist essentially of one or more compounds of Formula (I), one or more compounds of Formula (II), or combinations thereof. In some embodiments, the composition can be an organic composition without aqueous components.

In some embodiments, the vehicle can be a medium chain triglyceride (MCT). Medium-chain triglycerides (MCTs) are triglycerides of fatty acids. The fatty acids have an aliphatic chain of 6-12 carbon atoms, and can be, for example, hexanoic acid, octanoic acid, decanoic acid, and dodecanoic acid. The MCTs can be a single triglyceride or a mixture of triglycerides. Representative chemical structures of the MCTs are shown below:

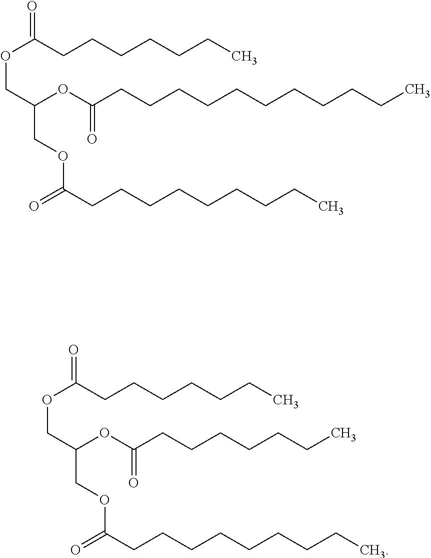 [SMILES: CCCCCCCCCCCC(=O)OC(COC(=O)CCCCCCC)COC(=O)CCCCCCCCC ; CCCCCCCCCC(=O)OCC(COC(=O)CCCCCCC)OC(=O)CCCCCCC]

In some embodiments, the MCT can be a triglyceride of fatty acids, and the fatty acids can be selected from the group consisting of hexanoic acid, octanoic acid, decanoic acid, dodecanoic acid, and combinations thereof.

In some embodiments, the MCT can be present in an amount of from about 0% to about 99.9% (w/w), from about 0.1% to about 99.9% (w/w), from about 0.1% to about 30% (w/w), from about 1% to about 25% (w/w), from about 1% to about 20% (w/w), from about 5% to about 20% (w/w), from about 5% to about 15% (w/w), from about 10% to about 99.9% (w/w), from about 20% to about 99.9% (w/w), from about 30% to about 99.9% (w/w), from about 40% to about 99.9% (w/w), from about 50% to about 99.9% (w/w), from about 60% to about 99.9% (w/w), from about 65% to about 99.9% (w/w), from about 70% to about 99.9% (w/w), from about 75% to about 99.9% (w/w), from about 80% to about 99.9% (w/w), from about 85% to about 99.9% (w/w), from about 90% to about 99.9% (w/w), from about 95% to about 99.9% (w/w), from about 98% to about 99.9% (w/w), from about 20% to about 80% (w/w), from about 20% to about 70% (w/w), from about 20% to about 60% (w/w), from about 20% to about 50% (w/w), from about 20% to about 40% (w/w), from about 20% to about 30% (w/w), from about 30% to about 80% (w/w), from about 40% to about 80% (w/w), from about 50% to about 80% (w/w), from about 60% to about 80% (w/w), from about 70% to about 80% (w/w), about 99.9% (w/w), about 99% (w/w), about 98% (w/w), about 97% (w/w), about 96% (w/w), about 95% (w/w), about 94% (w/w), about 93% (w/w), about 92% (w/w), about 91% (w/w), about 90% (w/w), about 85% (w/w), about 80% (w/w), about 75% (w/w), about 70% (w/w), about 65% (w/w), about 60% (w/w), about 55% (w/w), about 50% (w/w), about 45% (w/w), about 40% (w/w), about 35% (w/w), about 30% (w/w), about 25% (w/w), about 20% (w/w), about 15% (w/w), about 10% (w/w), about 9% (w/w), about 8% (w/w), about 7% (w/w), about 6% (w/w), about 5% (w/w), about 4% (w/w), about 3% (w/w), about 2% (w/w), about 1% (w/w), about 0.5% (w/w), or about 0.1% (w/w).

In some embodiments, the vehicle can be a semifluorinated alkane. A semifluorinated alkane is an amphiphilic liquid with two mutually immiscible moieties (hydrocarbon segment and perfluorinated segment) bound covalently. In one example, a partially fluorinated alkane compound useful in the formulations of this disclosure has a formula CF₃(CF₂)ₘ(CH₂)ₙCH₃. In some embodiments, m is an integer from 1 to 10, and n is an integer from 1 to 10. For example, m can be 1, 2, 3, 4, 5, or 6. In another example, n can be 1, 2, 3, 4, 5, or 6. Examples of Semifluorinated alkanes include perfluorobutylbutane (F4H4), perfluorobutylpentane (F4H5), perfluorobutylhexane (F4H6), perfluorohexylethane (F6H2), perfluorohexylbutane (F6H4), perfluorohexyloctane (F6H8), and perfluorohexyldecane (F6H10); preferably, perfluorohexylhexane (F6H6), and perfluorohexyloctane (F6H8).

### As an Example, the Structure of F6H8 is:

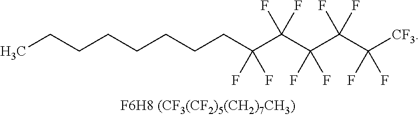 [SMILES: CCCCCCCCC(F)(F)C(F)(F)C(F)(F)C(F)(F)C(F)(F)C(F)(F)F]

In some embodiments, the semifluorinated alkane compound can have a formula of RFRH or a formula of RFRHRF; RF is a perfluorinated hydrocarbon with 1 to 15 carbon atoms, and RH is a non-fluorinated hydrocarbon with 1 to 15 carbon atoms.

In some embodiments, the concentration of the compound of Formula (I) or Formula (II) in a pharmaceutical composition comprising a partially fluorinated alkane vehicle liquid can be from about 1 mg/mL to about 10 mg/mL. For example, the concentration can be about 1 mg/mL, about 2 mg/mL, about 3 mg/mL, about 4 mg/mL, about 5 mg/mL, about 6 mg/mL, or about 7 mg/mL. In one example, the composition comprising a semifluorinated alkane fluid as a pharmaceutically acceptable vehicle can be a topical ophthalmic composition.

In some embodiments, the semifluorinated alkane can be present in an amount of from about 0% to about 99.9% (w/w), from about 0.1% to about 99.9% (w/w), from about 0.1% to about 30% (w/w), from about 1% to about 25% (w/w), from about 1% to about 20% (w/w), from about 5% to about 20% (w/w), from about 5% to about 15% (w/w), from about 10% to about 99.9% (w/w), from about 20% to about 99.9% (w/w), from about 30% to about 99.9% (w/w), from about 40% to about 99.9% (w/w), from about 50% to about 99.9% (w/w), from about 60% to about 99.9% (w/w), from about 65% to about 99.9% (w/w), from about 70% to about 99.9% (w/w), from about 75% to about 99.9% (w/w), from about 80% to about 99.9% (w/w), from about 85% to about 99.9% (w/w), from about 90% to about 99.9% (w/w), from about 95% to about 99.9% (w/w), from about 98% to about 99.9% (w/w), from about 20% to about 80% (w/w), from about 20% to about 70% (w/w), from about 20% to about 60% (w/w), from about 20% to about 50% (w/w), from about 20% to about 40% (w/w), from about 20% to about 30% (w/w), from about 30% to about 80% (w/w), from about 40% to about 80% (w/w), from about 50% to about 80% (w/w), from about 60% to about 80% (w/w), from about 70% to about 80% (w/w), about 99.9% (w/w), about 99% (w/w), about 98% (w/w), about 97% (w/w), about 96% (w/w), about 95% (w/w), about 94% (w/w), about 93% (w/w), about 92% (w/w), about 91% (w/w), about 90% (w/w), about 85% (w/w), about 80% (w/w), about 75% (w/w), about 70% (w/w), about 65% (w/w), about 60% (w/w), about 55% (w/w), about 50% (w/w), about 45% (w/w), about 40% (w/w), about 35% (w/w), about 30% (w/w), about 25% (w/w), about 20% (w/w), about 15% (w/w), about 10% (w/w), about 9% (w/w), about 8% (w/w), about 7% (w/w), about 6% (w/w), about 5% (w/w), about 4% (w/w), about 3% (w/w), about 2% (w/w), about 1% (w/w), about 0.5% (w/w), or about 0.1% (w/w).

In some embodiments, the vehicle can be a combination of one or more MCTs as described herein and one or more SFAs as described herein. In some embodiments, the MCT can be present in an amount of about 1% (w/w) and the SFA can be present in an amount of about 99% (w/w); the MCT can be present in an amount of about 2% (w/w) and the SFA can be present in an amount of about 98% (w/w); the MCT can be present in an amount of about 5% (w/w) and the SFA can be present in an amount of about 95% (w/w); the MCT can be present in an amount of about 10% (w/w) and the SFA can be present in an amount of about 90% (w/w); the MCT can be present in an amount of about 15% (w/w) and the SFA can be present in an amount of about 85% (w/w); the MCT can be present in an amount of about 20% (w/w) and the SFA can be present in an amount of about 80% (w/w); the MCT can be present in an amount of about 25% (w/w) and the SFA can be present in an amount of about 75% (w/w); the MCT can be present in an amount of about 30% (w/w) and the SFA can be present in an amount of about 70% (w/w); the MCT can be present in an amount of about 35% (w/w) and the SFA can be present in an amount of about 65% (w/w); the MCT can be present in an amount of about 40% (w/w) and the SFA can be present in an amount of about 60% (w/w); the MCT can be present in an amount of about 45% (w/w) and the SFA can be present in an amount of about 55% (w/w); the MCT can be present in an amount of about 50% (w/w) and the SFA can be present in an amount of about 50% (w/w); the MCT can be present in an amount of about 55% (w/w) and the SFA can be present in an amount of about 45% (w/w); the MCT can be present in an amount of about 60% (w/w) and the SFA can be present in an amount of about 40% (w/w); the MCT can be present in an amount of about 65% (w/w) and the SFA can be present in an amount of about 35% (w/w); the MCT can be present in an amount of about 70% (w/w) and the SFA can be present in an amount of about 30% (w/w); the MCT can be present in an amount of about 75% (w/w) and the SFA can be present in an amount of about 25% (w/w); the MCT can be present in an amount of about 80% (w/w) and the SFA can be present in an amount of about 20% (w/w); the MCT can be present in an amount of about 85% (w/w) and the SFA can be present in an amount of about 15% (w/w); the MCT can be present in an amount of about 90% (w/w) and the SFA can be present in an amount of about 10% (w/w); the MCT can be present in an amount of about 95% (w/w) and the SFA can be present in an amount of about 5% (w/w); the MCT can be present in an amount of about 98% (w/w) and the SFA can be present in an amount of about 2% (w/w); the MCT can be present in an amount of about 99% (w/w) and the SFA can be present in an amount of about 1% (w/w); MCT can be the only vehicle and the MCT can be present in an amount of about 99% (w/w); MCT can be the only vehicle and the MCT can be present in an amount of about 99.5% (w/w); MCT can be the only vehicle and the MCT can be present in an amount of about 99.6% (w/w); MCT can be the only vehicle and the MCT can be present in an amount of about 99.7% (w/w); MCT can be the only vehicle and the MCT can be present in an amount of about 99.8% (w/w); MCT can be the only vehicle and the MCT can be present in an amount of about 99.9% (w/w); SFA can be the only vehicle and the SFA can be present in an amount of about 99% (w/w); SFA can be the only vehicle and the SFA can be present in an amount of about 99.5% (w/w); SFA can be the only vehicle and the SFA can be present in an amount of about 99.6% (w/w); SFA can be the only vehicle and the SFA can be present in an amount of about 99.7% (w/w); SFA can be the only vehicle and the SFA can be present in an amount of about 99.8% (w/w); or SFA can be the only vehicle and the SFA can be present in an amount of about 99.9% (w/w).

The compositions disclosed herein can be pharmaceutical compositions comprising an effective amount of a compound of Formula (I), Formula (II), or a pharmaceutically acceptable salts thereof, or combinations thereof; and a pharmaceutically acceptable carrier or vehicle as described herein. In some embodiments, the compositions can be topical compositions. In some embodiments, the compositions can be topical ocular compositions.

The pharmaceutical compositions may also comprise one or more additional therapeutic agents, excipients, or diluents including, but not limited to, absorbents, anti-irritants, preservatives, antioxidants, coloring agents/pigments, emollients (moisturizers), emulsifiers, film-forming/holding agents, prescription drugs, surfactants/detergent cleansing agents, penetration enhancers, viscosity enhancers, and thickeners.

The pharmaceutical compositions of the present application can include those suitable for any acceptable route of administration. Suitable examples of acceptable routes of administration include topical, ocular, topical ocular, injection, and the like.

Compositions and formulations described herein can be conveniently presented in a unit dosage form or a multi-dosage form. In some embodiments, compositions and formulations described herein can be conveniently presented in a dosage form (unit or multiple) that can be used with or in an automated dosing apparatus or regimen.

In some embodiments, the compositions are prepared by uniformly and intimately bringing into association the compounds of Formula (I) or Formula (II) as disclosed herein with the liquid vehicles disclosed herein.

In some embodiments, the topical compositions of the present disclosure can be prepared and used in the form of an aerosol spray, cream, emulsion, solid, liquid, dispersion, foam, oil, gel, hydrogel, lotion, mousse, ointment, pomade, or solution. In some embodiments, the composition is in any form commonly employed in the art of cosmetic and skin care formulation. In some embodiments, the topical compositions can be in an emulsion form. In some embodiments, the topical compositions can be in an organic solution. In some embodiments, the compositions can be in the form of an organic composition without aqueous components.

In some embodiments, the topical composition is an ophthalmic composition, e.g., for intraocular administration. Suitable examples of ophthalmic compositions include eye drops, suspensions, ointments, emulsions, nanoemulsions, oils, gels, hydrogels, and solutions. Any one of these ophthalmic compositions can be administered directly to the cornea, using, for example, a plastic or latex applicator (e.g., a single-use applicator), an eye dropper, a glass pipette, or a rubber bulb. These compositions may contain, for example, a semifluorinated alkane liquid vehicle, or a MCT liquid vehicle, or combinations thereof as described above.

In some embodiments, the topical composition is a cosmetic composition, e.g., for topical administration to e.g., epidermal tissue, nails, or hair. Suitable examples of cosmetic compositions include suspensions, ointments, emulsions, nanoemulsions, oils, gels, creams, mousse, sprays, hydrogels, and solutions. Any one of these cosmetic compositions can be administered directly to the skin, hair, or nails, using, for example, a plastic or latex applicator (e.g., a single-use applicator), a dropper, a cotton or fabric applicators, a sponge, a glass pipette, or a rubber bulb. These compositions may contain, for example, a semifluorinated alkane liquid vehicle, or a MCT liquid vehicle, or combinations thereof as described above.

This disclosure also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent as described herein.

In some embodiments, a kit can comprise a dispensing device preloaded with a composition as described herein.

This disclosure also provides methods for crosslinking structural proteins using the compounds and compositions described herein. Structural proteins that can be photo-crosslinked using the compounds, compositions, or methods described herein can include any structural proteins that are photo-crosslinkable in the presence of a reactive oxygen facilitator. Exemplary structural proteins include, without limitation, collagens and collagen derivatives, keratins and keratin derivatives, elastins and elastin derivatives, and the like. As used herein, “collagen” includes, without limitation, collagen fibers or fibrils. As used herein, “keratin” includes, without limitation, keratin fibers or fibrils.

In some embodiments, the methods can comprise contacting the structural proteins or a biological tissue comprising the structural proteins with an effective amount of a compound of Formula (I) or Formula (II) or combinations thereof, or with an effective amount of a composition comprising Formula (I) or Formula (II) or combinations thereof; and irradiating the structural proteins or the tissue comprising the structural proteins.

In some embodiments, methods are provided for crosslinking collagen in a tissue of a subject. In some embodiments, the methods can comprise administering to the tissue of the subject an effective amount of a compound of Formula (I) or Formula (II) or combinations thereof, or with an effective amount of a composition comprising Formula (I) or Formula (II) or combinations thereof; and irradiating the tissue.

In some embodiments, the irradiation can include irradiating with UV light (e.g., UVA light), or with blue light. In some embodiments, contacting the structural proteins or a biological tissue comprising the structural proteins can include, without limitation, topically applying the compounds or compositions to the proteins or tissues, immersing the proteins or tissues in the compositions, spraying the proteins or tissues with the compounds or compositions, injecting the compounds or compositions into the tissue or into a matrix comprising the structural proteins, and the like. In some embodiments, contacting the structural proteins or a biological tissue comprising the structural proteins can include administering an effective amount of a compound of Formula (I) or Formula (II) or combinations thereof, or an effective amount of a composition comprising Formula (I) or Formula (II) or combinations thereof, to a tissue in the body of a subject.

Effective amounts of a compound of Formula (I) or Formula (II) or combinations thereof, or an effective amount of a composition comprising Formula (I) or Formula (II) or combinations thereof can be administered to various bodily tissues or biological tissues. In some embodiments, exemplary bodily tissues or biological tissues can include, without limitation, collagenous tissues, keratinous tissues, corneal tissues, epithelial tissues, epidermal tissues, pericardial tissues, skin, hair, or nails. In some embodiments, the tissues can be in or on the body of a subject. In some embodiments, the tissues can be ex vivo or in vitro. In some embodiments, the tissues can be bovine or porcine tissues.

Also provided herein are methods for treating ocular diseases or conditions using the compounds, compositions, and methods described herein. In some embodiments, the ocular diseases or conditions can be related to the cornea or corneal tissues. Exemplary, nonlimiting ocular diseases that can be treated with the compounds, compositions, and methods described herein include chemical burns or a disease selected from keratoconus, thin corneas, keratitis, infectious keratitis including bacterial or fungal keratitis, corneal ulcer, bullous keratopathy and other forms of corneal edema, keratolysis, an autoimmune disease, a cicatrizing disease such as Ocular Cicatricial Pemphigoid, Stevens-Johnson Syndrome, Lupus Erythematosus, Rheumatoid Arthritis, corneal ectasia including cornea ectasia appearing after laser-assisted in situ keratomileusis (LASIK) and photorefractive keratectomy (PRK), post-refractive ectasia, corneal degeneration, brittle cornea syndrome, Ehlers-Danlos Syndrome Type VI, corneal neovascularization, corneal melting including Corneal Melting in Boston Keratoprosthesis Type I, hyperopia, hyperopic astigmatism, myopia, astigmatism, myopic astigmatism, myopic regression, keratopathy, corneal astigmatism, unstable cornea, or pellucid marginal degeneration (PMD).

In some embodiments, methods are provided for administering, to the eye and/or corneal tissue of a subject, an effective amount of a compound of Formula (I) or Formula (II) or combinations thereof, or with an effective amount of a composition comprising Formula (I) or Formula (II) or combinations thereof; and irradiating at least a portion of the eye and/or corneal tissue with, e.g., UV light or blue light. In some embodiments, the compounds of Formula (I) or Formula (II) or combinations thereof, or with the compositions comprising Formula (I) or Formula (II) or combinations thereof can be in the form of an eye drop. In some embodiments, the methods can further comprise applying the eye drop to an eye of the subject. In some embodiments, the methods can further comprise de-epithelializing at least a portion of the cornea. In some embodiments, the crosslinking method can be performed without de-epithelializing any portion of the cornea.

The compounds, compositions, and methods described herein can, in some embodiments, be useful for crosslinking structural proteins in biological tissues or other formats ex vivo. Exemplary ex vivo applications include, without limitation compositions, biomaterials, biological matrices, and biological tissues for experimental or in vitro use or for biomaterials, tissues, hydrogels, and matrices intended for wound healing, tissue regeneration, tissue engineering, or drug delivery applications, and, for example, compositions, biological matrices, and biological tissues intended for use in hydrogels, biological scaffolds, surgical implants, bioprosthetics, grafts, and transplants and the like. Exemplary biomaterials include, without limitation, natural or synthetic variants of heart valves, corneal transplants, arterial prostheses, arterial grafts, wound healing matrices or grafts, skin grafts, vascular grafts, transdermal drug delivery devices or patches, and the like. In some embodiments, these biomaterials can also be subject to photo-crosslinking using the compounds, compositions, and methods described herein within or on the body of a subject, for example, during or after delivery of the biomaterials to the subject such as through surgery or other delivery methods.

The invention will be further described in the following examples, which do not limit the scope of the invention.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### EXAMPLES

### Example 1a—Solubility of Riboflavin in Organic Solvents

Solubility of riboflavin was tested in medium chain triglycerides (MCT), F6H8, or mixtures of MCT/F6H8 at 100° C. Solubility was determined by HPLC according to the conditions outlined in Table 1.

TABLE 1
Chromatographic Conditions

| Column: | Nano Chrom Core 120, C18 ,4.6*250 mm, 5 μm | | |
|---|---|---|---|
| Mobile phase A: | 0.5% acetic acid in (water:acetonitrile = | | |
| | 80:20) solution(v/v) | | |
| Mobile phase B: | methanol | | |
| Diluent: | methanol | | |
| Flow rate: | 1.0 mL/min | | |
| Injection volume: | 5 μL | | |
| Needle washing | 50:50 Acetonitrile:Deionized water | | |
| solution: | | | |
| Column temperature: | 40° C. | | |
| Automatic sampler | 20° C. | | |
| temperature: | | | |
| Detection wavelength: | UV detector, 268 nm | | |
| Running time: | 25 min | | |
| | Time | % Mobile | % Mobile |
| | (min) | phase A | phase B |
| Gradient: | 0.00 | 80 | 20 |
| | 10.00 | 20 | 80 |
| | 20 | 20 | 80 |
| | 20.01 | 80 | 20 |
| | 25 | 80 | 20 |

### The Results are Shown in Table 1a Below:

TABLE 1a
Solubility of Riboflavin in Organic Solvents

| | | | | | Solubility |
|---|---|---|---|---|---|
| Sample No. | Compound | Temp. | MCT | F6H8 | (μg/mL) |
| A1 | RTB | 100° C. | 0% | 100% | 0.000 |
| A2 | RTB | 100° C. | 100% | 0% | 0.000 |
| A3 | RTB | 25° C. | 10% | 90% | 0.000 |

### Example 1b—Solubility of Riboflavin Tetrabutyrate in MCT, F6H8, or Mixed Solvents

Solubility of riboflavin tetrabutyrate (RTB) was tested in medium chain triglycerides (MCT), F6H8, or mixtures of MCT/F6H8 at room temperature, and in F6H8 at 100° C. Solubility was analyzed as in Example 1a.

### The Results are Shown in Table 1b Below:

TABLE 1b
Solubility of RTB in Organic Solvents

| | | | | | Solubility |
|---|---|---|---|---|---|
| Sample No. | Compound | Temp. | MCT | F6H8 | (μg/mL) |
| B1 | RTB | 100° C. | 0% | 100% | 0.000 |
| B2 | RTB | 25° C. | 100% | 0% | 3.133 |
| B3 | RTB | 25° C. | 20% | 80% | 0.316 |
| B4 | RTB | 25° C. | 50% | 50% | 1.124 |
| B5 | RTB | 25° C. | 70% | 30% | 1.582 |

### Example 1c—Aqueous Solubility of Riboflavin Tetrabutyrate

Solubility of riboflavin tetrabutyrate (RTB) was tested in PBS solution, with or without 10% hydroxypropyl-beta-cyclodextrin (HP-β-CD) as solubilizer, at varying pH. For each formulation, 5 mg of RTB was added to 5 mL of pH-adjusted PBS containing 10% HP-β-CD at 25° C., and mixed well. Solubility was analyzed as in Example 1a. The results are shown in Table 1c below:

TABLE 1c
Saturated Aqueous Solubility of RTB

| | | | | | Solubility |
|---|---|---|---|---|---|
| Sample No. | Compound | Temp. | HP-β-CD | pH | (μg/mL) |
| C1 | RTB | 25° C. | 10% | 5.0 | 0.008 |
| C2 | RTB | 25° C. | 10% | 5.5 | 0.003 |
| C3 | RTB | 25° C. | 10% | 6.0 | 0.004 |
| C4 | RTB | 25° C. | — | 5.0 | 0.008 |
| C5 | RTB | 25° C. | — | 5.5 | 0.001 |
| C6 | RTB | 25° C. | — | 6.0 | 0.002 |

### Example 2—Stability of Riboflavin Tetrabutyrate in Organic Solvents

The stability of riboflavin tetrabutyrate (RTB) in various solvents was investigated. Using similar sample analysis methods described in Examples 1a-1c, the stability of RTB in formulations of MCT, F6H8, and combinations were monitored at various temperatures. The results are summarized in Table 2 below.

TABLE 2
Stability of Riboflavin Tetrabutyrate in Organic Solvents

| | | | | | | Conc. | Conc. | Conc. |
|---|---|---|---|---|---|---|---|---|
| Sample | Compound/ | | | | | (mg/mL) | (mg/mL) | (mg/mL) |
| No. | Concentration | MCT | F6H8 | Temp. | | 0 hr | 14 days | 1 Month |
| X1 | RTB, 0.2% | 100% | 0% | 2-8° | C. | 2.055, | 1.837, | 1.832, |
| | | | | | | clear | clear | clear |
| X2 | RTB, 0.2% | 100% | 0% | 25° | C. | 2.055, | 1.888, | 1.817, |
| | | | | | | clear | clear | clear |
| X3 | RTB, 0.2% | 100% | 0% | 40° | C. | 2.055, | 1.958, | 1.833, |
| | | | | | | clear | clear | clear |
| X4 | RTB, 0.1% | 70% | 30% | 2-8° | C. | 1.077, | 1.015, | 0.960, |
| | | | | | | clear | clear | clear |
| X5 | RTB, 0.1% | 70% | 30% | 25° | C. | 1.077, | 1.012, | 0.953, |
| | | | | | | clear | clear | clear |
| X6 | RTB, 0.1% | 70% | 30% | 40° | C. | 1.077, | 1.027, | 0.962, |
| | | | | | | clear | clear | clear |
| X7 | RTB, 0.1% | 50% | 50% | 2-8° | C. | 1.123, | 0.976, | 0.848, |
| | | | | | | clear | precip. | precip. |
| X8 | RTB, 0.1% | 50% | 50% | 25° | C. | 1.123, | 1.056, | 0.993, |
| | | | | | | clear | clear | clear |
| X9 | RTB, 0.1% | 50% | 50% | 40° | C. | 1.123, | 1.054, | 0.984, |
| | | | | | | clear | clear | clear |

### Example 3a—Impurities from Riboflavin Tetrabutyrate in Aqueous Solution

The impurities present in PBS solutions, with or without 10% hydroxy-propyl-beta-cyclodextrin (HP-β-CD) as solubilizer, at varying pH, with riboflavin tetrabutyrate (RTB) was investigated. Using similar sample analysis methods described in Examples 1a-1c, the impurities were determined. The results are summarized in Table 3a below.

TABLE 3a
Impurities from Riboflavin Tetrabutyrate in Aqueous Solution

| Sample | | | Riboflavin | Other | |
|---|---|---|---|---|---|
| No. | HP-β-CD | pH | Tetrabutyrate | Impurities | Riboflavin |
| YA1 | 10% | 5.0 | 96.28% | 2.94% | 0% |
| YA2 | 10% | 6.0 | 87.2% | 10.65% | 2.45% |
| YA3 | 10% | 7.0 | 88.58% | 9.39% | 2.03% |
| YA4 | — | 5.0 | 70.22% | 22.54% | 7.24% |
| YA5 | — | 6.0 | 71.90% | 20.58% | 7.52% |
| YA6 | — | 7.0 | 71.30% | 19.8% | 8.92% |

### Example 3b—Impurities from Riboflavin Tetrabutyrate in Organic Solvents

The impurities present in MCT, F6H8, or combinations, with riboflavin tetrabutyrate (RTB), at 25° C. was investigated. Using similar sample analysis methods described in Examples 1a-1c, the impurities were determined. The results are summarized in Table 3b below.

TABLE 3b
Impurities from Riboflavin Tetrabutyrate in Organic Solvents

| Sample | Cpd./ | | | 0 hr | | 14 Days | | 1 Month | |
|---|---|---|---|---|---|---|---|---|---|
| No. | Conc. | MCT | F6H8 | RTB | Impur. | RTB | Impur. | RTB | Impur. |
| YB1 | RTB, | 100% | 0% | 98.76% | 1.16% | 98.76% | 1.16% | 98.71% | 1.15% |
| | 0.2% | | | | | | | | |
| YB2 | RTB, | 70% | 30% | 98.85% | 1.14% | 98.94% | 1.06% | 99.05% | 0.95% |
| | 0.1% | | | | | | | | |
| YB3 | RTB, | 50% | 50% | 98.87% | 1.13% | 98.92% | 1.07% | 98.99% | 1.02% |
| | 0.1% | | | | | | | | |

### Example 3—Ex Vivo Study (Dresden Method)

Based on Gregor Wollensak, Eberhard Spoerl, Theo Seiler. Riboflavin/ultraviolet-a-induced collagen crosslinking for the treatment of keratoconus. Am J Ophthalmol. 2003 May; 135 (5): 620-7

Fresh porcine cadaver eyes with intact epithelia and clear corneas are retrieved from the local slaughterhouse within 2 to 5 hours post-mortem. The eyes are de-epithelialized mechanically, removing the corneoscleral ring with scissors. With a self-constructed double-blade scalpel, 1 corneal strip of 5.0 mm width, 850 μm central corneal thickness, and 14.0 mm length including 1.0 mm sclera on both ends is cut in a superior-inferior fashion from the 12 o'clock position of the cornea, which is identified by its oval shape. Because of the natural thickness of the porcine cornea, only 1 corneal strip with clearly cut perpendicular edges could be prepared properly from each eye. Half of the corneas are treated with RTB-UVA irradiation, and half are used as untreated controls. Using ultrasound pachymetry (Pachette, Technomed), the central corneal thickness is determined in the eyes.

### Treatment

Starting 5 minutes before the treatment, 0.1% riboflavin tetrabutyrate (RTB) photosensitizer solution (10 mg riboflavin tetrabutyrate in 10 mL MCT or F6H8 or a combination thereof) is dropped on the treated strips and MCT or F6H8 or combination solution is dropped on the control strips at 5-minute intervals. Ultraviolet A irradiation (370 nm) is applied using 2 double UVA diodes (Roithner Lasertechnik) with an irradiance of 3 mW/cm2 at a distance of 1.0 cm from the cornea for 30 minutes. This is equal to a dose of 5.4 J/cm². The parameters of exposure are chosen according to the treatment procedure used in keratoconus patients. Three 1.3 V accumulators are used as a power generator. Before treatment, the desired irradiance of 3 mW/cm2 is controlled with a calibrated UVA meter (LaserMate-Q, Laser2000) at a distance of 1.0 cm and, if necessary, regulated with a potentiometer.

### Static Stress-Strain Measurements

The porcine corneal strips are clamped horizontally at a distance of 8.0 mm between the jaws of a commercially available microcomputer-controlled biomaterial tester (Minimat, Rheometric Scientific GmbH. To include the physiological stress range, a prestress of 5×10³ Pa (1 Pa=1 N/m2) is used, which requires a force of 20 mN in porcine corneas. The strain is then increased linearly with a velocity of 1.5 mm min-1, and the stress is measured up to 2×10⁵ Pa. The stress-strain values are fitted by an exponential function σ=A exp (BX) using the SPSS-calculation program (SPSS GmbH Software, Munich). Young's modulus (E) is calculated for 4%, 6%, and 8% strain as the gradient of the stress-strain graph (E=dσ/dε=A×B exp (B×ε).

### Statistical Evaluation

The stress data necessary for a strain of 4%, 6%, and 8% in treated and untreated corneas is compared using the Student t-test.

### Result

### Stress-Strain Curves

The stress-strain curves showed the typical exponential increase of a bioviscoelastic solid. In porcine corneas, the stress using strain is greater in the treated corneas and lesser in the untreated corneas, corresponding to an increase in the treated corneas.

### Without De-Epithelialization

Similar experimentation is repeated for intact porcine eyes except that the corneas are not de-epithelialized. RTB formulations in MCT or F6H8 are applied to half of the intact eyes and these are treated with UVA radiation. Following the application and treatment, 1 corneal strip is retrieved from each eye, treated and non-treated, according to the procedure described above. Corneal thickness and stress/strain measurements are obtained according to the procedure described above. In porcine corneas, the stress using strain is greater in the treated corneas and lesser in the untreated corneas, corresponding to an increase in the treated corneas.

### Other Embodiments

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

Embodiment 1 is composition comprising:
a compound of Formula I:

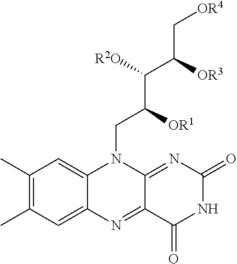

wherein:
   R1, R2, and R3 are each independently selected from hydrogen or —C(O)R⁵;
   R4 is selected from hydrogen, —C(O)R⁵, a phosphate, or a salt thereof; and
R5 is a linear or branched or cyclic alkyl having 1 to 26 carbon atoms; and
a vehicle selected from a medium chain triglyceride (MCT), a semifluorinated alkane, or combinations thereof.

Embodiment 2 is the composition of Embodiment 1, wherein the MCT is a triglyceride of fatty acids, and the fatty acids are selected from the group consisting of hexanoic acid, octanoic acid, decanoic acid, dodecanoic acid, and combinations thereof.

Embodiment 3 is the composition of any one of Embodiments 1 or 2, wherein the semifluorinated alkane compound has a formula of RFRH or a formula of RFRHRF; RF is a perfluorinated hydrocarbon with 1 to 15 carbon atoms, and wherein RH is a non-fluorinated hydrocarbon with 1 to 15 carbon atoms.

Embodiment 4 is the composition of any one of Embodiments 1 to 3, wherein the semifluorinated alkane is selected from perfluorobutylbutane (F4H4), perfluorobutylpentane (F4H5), perfluorobutylhexane (F4H6), perfluorohexylethane (F6H2), perfluorohexylbutane (F6H4), perfluorohexyloctane (F6H8), and perfluorohexyldecane (F6H10); preferably, perfluorohexylhexane (F6H6), and perfluorohexyloctane (F6H8).

Embodiment 5 is the composition of any one of Embodiments 1 to 4, wherein the semifluorinated alkane is perfluorohexyloctane (F6H8).

Embodiment 6 is the composition of any one of Embodiments 1 to 5, wherein the compound of Formula I is present in an amount of from about 0.001% to about 1% (w/w), from about 0.01% to about 1% (w/w), from about 0.05% to about 0.5% (w/w), about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), or 1% (w/w).

Embodiment 7 is the composition of any one of Embodiments 1 to 6, wherein the MCT is present in an amount of from about 0% to about 99.9% (w/w), from about 0% to about 80% (w/w), from about 0.1% to about 99.9% (w/w), from about 0.1% to about 30% (w/w), from about 1% to about 25% (w/w), from about 1% to about 20% (w/w), from about 5% to about 20% (w/w), from about 5% to about 15% (w/w), from about 10% to about 99.9% (w/w), from about 20% to about 99.9% (w/w), from about 30% to about 99.9% (w/w), from about 40% to about 99.9% (w/w), from about 50% to about 99.9% (w/w), from about 60% to about 99.9% (w/w), from about 65% to about 99.9% (w/w), from about 70% to about 99.9% (w/w), from about 75% to about 99.9% (w/w), from about 80% to about 99.9% (w/w), from about 85% to about 99.9% (w/w), from about 90% to about 99.9% (w/w), from about 95% to about 99.9% (w/w), from about 98% to about 99.9% (w/w), from about 20% to about 80% (w/w), from about 20% to about 70% (w/w), from about 20% to about 60% (w/w), from about 20% to about 50% (w/w), from about 20% to about 40% (w/w), from about 20% to about 30% (w/w), from about 30% to about 80% (w/w), from about 40% to about 80% (w/w), from about 50% to about 80% (w/w), from about 60% to about 80% (w/w), from about 70% to about 80% (w/w), about 99.9% (w/w), about 99% (w/w), about 98% (w/w), about 97% (w/w), about 96% (w/w), about 95% (w/w), about 94% (w/w), about 93% (w/w), about 92% (w/w), about 91% (w/w), about 90% (w/w), about 85% (w/w), about 80% (w/w), about 75% (w/w), about 70% (w/w), about 65% (w/w), about 60% (w/w), about 55% (w/w), about 50% (w/w), about 45% (w/w), about 40% (w/w), about 35% (w/w), about 30% (w/w), about 25% (w/w), about 20% (w/w), about 15% (w/w), about 10% (w/w), about 9% (w/w), about 8% (w/w), about 7% (w/w), about 6% (w/w), about 5% (w/w), about 4% (w/w), about 3% (w/w), about 2% (w/w), about 1% (w/w), about 0.5% (w/w), or about 0.1% (w/w).

Embodiment 8 is the composition of any one of Embodiments 1 to 7, wherein the semifluorinated alkane is present in an amount of from about 0% to about 99.9% (w/w), from about 0.1% to about 99.9% (w/w), from about 0.1% to about 30% (w/w), from about 1% to about 25% (w/w), from about 1% to about 20% (w/w), from about 5% to about 20% (w/w), from about 5% to about 15% (w/w), from about 10% to about 99.9% (w/w), from about 20% to about 99.9% (w/w), from about 30% to about 99.9% (w/w), from about 40% to about 99.9% (w/w), from about 50% to about 99.9% (w/w), from about 60% to about 99.9% (w/w), from about 65% to about 99.9% (w/w), from about 70% to about 99.9% (w/w), from about 75% to about 99.9% (w/w), from about 80% to about 99.9% (w/w), from about 85% to about 99.9% (w/w), from about 90% to about 99.9% (w/w), from about 95% to about 99.9% (w/w), from about 98% to about 99.9% (w/w), from about 20% to about 80% (w/w), from about 20% to about 70% (w/w), from about 20% to about 60% (w/w), from about 20% to about 50% (w/w), from about 20% to about 40% (w/w), from about 20% to about 30% (w/w), from about 30% to about 80% (w/w), from about 40% to about 80% (w/w), from about 50% to about 80% (w/w), from about 60% to about 80% (w/w), from about 70% to about 80% (w/w), about 99.9% (w/w), about 99% (w/w), about 98% (w/w), about 97% (w/w), about 96% (w/w), about 95% (w/w), about 94% (w/w), about 93% (w/w), about 92% (w/w), about 91% (w/w), about 90% (w/w), about 85% (w/w), about 80% (w/w), about 75% (w/w), about 70% (w/w), about 65% (w/w), about 60% (w/w), about 55% (w/w), about 50% (w/w), about 45% (w/w), about 40% (w/w), about 35% (w/w), about 30% (w/w), about 25% (w/w), about 20% (w/w), about 15% (w/w), about 10% (w/w), about 9% (w/w), about 8% (w/w), about 7% (w/w), about 6% (w/w), about 5% (w/w), about 4% (w/w), about 3% (w/w), about 2% (w/w), about 1% (w/w), about 0.5% (w/w), or about 0.1% (w/w).

Embodiment 9 is the composition of any one of Embodiments 1 to 8, wherein the MCT is present in an amount of about 1% (w/w) and the SFA is present in an amount of about 99% (w/w), wherein the MCT is present in an amount of about 2% (w/w) and the SFA is present in an amount of about 98% (w/w), wherein the MCT is present in an amount of about 5% (w/w) and the SFA is present in an amount of about 95% (w/w), wherein the MCT is present in an amount of about 10% (w/w) and the SFA is present in an amount of about 90% (w/w), wherein the MCT is present in an amount of about 15% (w/w) and the SFA is present in an amount of about 85% (w/w), wherein the MCT is present in an amount of about 20% (w/w) and the SFA is present in an amount of about 80% (w/w), wherein the MCT is present in an amount of about 25% (w/w) and the SFA is present in an amount of about 75% (w/w), wherein the MCT is present in an amount of about 30% (w/w) and the SFA is present in an amount of about 70% (w/w), wherein the MCT is present in an amount of about 35% (w/w) and the SFA is present in an amount of about 65% (w/w), wherein the MCT is present in an amount of about 40% (w/w) and the SFA is present in an amount of about 60% (w/w), wherein the MCT is present in an amount of about 45% (w/w) and the SFA is present in an amount of about 55% (w/w), wherein the MCT is present in an amount of about 50% (w/w) and the SFA is present in an amount of about 50% (w/w), wherein the MCT is present in an amount of about 55% (w/w) and the SFA is present in an amount of about 45% (w/w), wherein the MCT is present in an amount of about 60% (w/w) and the SFA is present in an amount of about 40% (w/w), wherein the MCT is present in an amount of about 65% (w/w) and the SFA is present in an amount of about 35% (w/w), wherein the MCT is present in an amount of about 70% (w/w) and the SFA is present in an amount of about 30% (w/w), wherein the MCT is present in an amount of about 75% (w/w) and the SFA is present in an amount of about 25% (w/w), wherein the MCT is present in an amount of about 80% (w/w) and the SFA is present in an amount of about 20% (w/w), wherein the MCT is present in an amount of about 85% (w/w) and the SFA is present in an amount of about 15% (w/w), wherein the MCT is present in an amount of about 90% (w/w) and the SFA is present in an amount of about 10% (w/w), wherein the MCT is present in an amount of about 95% (w/w) and the SFA is present in an amount of about 5% (w/w), wherein the MCT is present in an amount of about 98% (w/w) and the SFA is present in an amount of about 2% (w/w), wherein the MCT is present in an amount of about 99% (w/w) and the SFA is present in an amount of about 1% (w/w), wherein MCT is the only vehicle and the MCT is present in an amount of about 99% (w/w), wherein MCT is the only vehicle and the MCT is present in an amount of about 99.5% (w/w), wherein MCT is the only vehicle and the MCT is present in an amount of about 99.6% (w/w), wherein MCT is the only vehicle and the MCT is present in an amount of about 99.7% (w/w), wherein MCT is the only vehicle and the MCT is present in an amount of about 99.8% (w/w), wherein MCT is the only vehicle and the MCT is present in an amount of about 99.9% (w/w), wherein SFA is the only vehicle and the SFA is present in an amount of about 99% (w/w), wherein SFA is the only vehicle and the SFA is present in an amount of about 99.5% (w/w), wherein SFA is the only vehicle and the SFA is present in an amount of about 99.6% (w/w), wherein SFA is the only vehicle and the SFA is present in an amount of about 99.7% (w/w), wherein SFA is the only vehicle and the SFA is present in an amount of about 99.8% (w/w), or wherein SFA is the only vehicle and the SFA is present in an amount of about 99.9% (w/w).

Embodiment 10 is the composition of any one of Embodiments 1 to 9, wherein:
R1, R2, R3, and R4 are each independently selected from hydrogen or —C(O)R⁵;
R5 is a linear or branched or cyclic alkyl having 1 to 26 carbon atoms; and
at least one of R1, R2, R3, and R4 is —C(O)R⁵, at least two of R1, R2, R3, and R4 is —C(O)R⁵, or at least three of R1, R2, R3, and R4 is —C(O)R⁵.

Embodiment 11 is the composition of any one of Embodiments 1 to 10, wherein:
R1, R2, R3, and R4 are each independently —C(O)R⁵; and
R5 is a linear or branched or cyclic alkyl having 1 to 26 carbon atoms. Embodiment 12 is the composition of any one of Embodiments 1 to 11, wherein:
R1, R2, R3, and R4 are each independently selected from hydrogen or —C(O)R⁵;
at least one of R1, R2, R3, and R4 is —C(O)R⁵, at least two of R1, R2, R3, and R4 is —C(O)R⁵, or at least three of R1, R2, R3, and R4 is —C(O)R⁵; and
R5 is selected from a linear or branched alkyl having 1 to 3 or 15 carbon atoms.

Embodiment 13 is the composition of any one of Embodiments 1 to 12, wherein R1, R2, R3, and R4 are each independently —C(O)R⁵, and wherein R5 is selected from a linear or branched alkyl having 1 to 3 or 15 carbon atoms.

Embodiment 14 is the composition of any one of Embodiments 1 to 12, wherein the compound of Formula I is selected from riboflavin tetraacetate, riboflavin tetrapropionate, riboflavin tetrabutyrate, riboflavin tetrapalmitate, or combinations thereof.

Embodiment 15 is the composition of any one of Embodiments 1 to 14, wherein the composition consists essentially of the compound of Formula I and the vehicle.

Embodiment 16 is the composition of any one of Embodiments 1 to 15, wherein the composition is an organic composition without aqueous components.

Embodiment 17 is the composition of any one of Embodiments 1 to 16, wherein the composition is a topical composition or a topical ocular composition, and wherein the composition is a solution, a suspension, or an emulsion.

Embodiment 18 is the composition of any one of Embodiments 1 to 17, wherein the composition further comprises a viscosity enhancer.

Embodiment 19 is a method for crosslinking collagen in a tissue of a subject, comprising:
administering to the tissue of the subject a compound of Formula II:

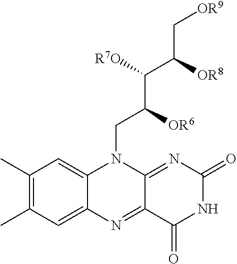

wherein:
   R6, R7, R8, and R9 are each independently selected from hydrogen or —C(O)R⁵;
   at least one of R6, R7, R8, and R9 is —C(O)R⁵; and
   R5 is selected from a linear or branched or cyclic alkyl
having 1 to 26 carbon atoms; and
irradiating the tissue with UV radiation or blue light.

Embodiment 20 is a method for crosslinking a structural protein, comprising:
contacting the structural protein or a biological tissue containing the structural protein with a compound of Formula II:

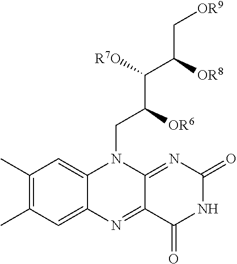

wherein:
   R6, R7, R8, and R9 are each independently selected from hydrogen or —C(O)R⁵;
   at least one of R6, R7, R8, and R9 is —C(O)R⁵; and
   R5 is selected from a linear or branched or cyclic alkyl having 1 to 26 carbon atoms; and
irradiating the structural protein or the tissue with UV radiation or blue light.

Embodiment 21 is the method of any one of Embodiments 19 or 20, wherein for Formula II:
R1, R2, R3, and R4 are each independently selected from hydrogen or —C(O)R⁵;
R5 is selected from a linear or branched or cyclic alkyl having 1 to 26 carbon atoms; and
at least one of R1, R2, R3, and R4 is —C(O)R⁵, at least two of R1, R2, R3, and R4 are —C(O)R⁵, or at least three of R1, R2, R3, and R4 are —C(O)R⁵. Embodiment 22 is the method of any one of Embodiments 19 to 21, wherein:
R1, R2, R3, and R4 are each independently —C(O)R⁵; and
R5 is selected from a linear or branched or cyclic alkyl having 1 to 26 carbon atoms.

Embodiment 23 is the method of any one of Embodiments 19 to 21, wherein:
R1, R2, R3, and R4 are each independently selected from hydrogen or —C(O)R⁵;
at least one of R1, R2, R3, and R4 is —C(O)R⁵, at least two of R1, R2, R3, and R4 are —C(O)R⁵, or at least three of R1, R2, R3, and R4 are —C(O)R⁵; and
R5 is selected from a linear or branched alkyl having 1 to 3 or 15 carbon atoms.

Embodiment 24 is the method of any one of Embodiments 19 to 23, wherein R1, R2, R3, and R4 are each independently —C(O)R⁵, and wherein R5 is selected from a linear or branched alkyl having 1 to 3 or 15 carbon atoms.

Embodiment 25 is the method of any one of Embodiments 19 to 23, wherein the compound of Formula II is selected from riboflavin tetraacetate, riboflavin tetrapropionate, riboflavin tetrabutyrate, riboflavin tetrapalmitate, or combinations thereof.

Embodiment 26 is the method of any one of Embodiments 19 to 25, wherein the structural protein is selected from collagen, keratin, derivatives thereof, and combinations thereof.

Embodiment 27 is the method of any one of Embodiments 19 to 26, wherein the structural protein is present in a biological tissue or is present in or on a body of a subject.

Embodiment 28 is the method of any one of Embodiments 19 to 27, wherein the biological tissue is ex vivo.

Embodiment 29 is the method of any one of Embodiments 19 to 27, wherein the biological tissue is in or on a body of a subject.

Embodiment 30 is the method of any one of Embodiments 19 to 29, wherein contacting the structural protein or contacting the tissue comprises administering the compound of Formula II to a tissue in a body of the subject.

Embodiment 31 is the method of any one of Embodiments 19 to 30, wherein the tissue is selected from a collagenous tissue, epidermal tissue, keratinous tissue, or combinations thereof.

Embodiment 32 is the method of claim 31, wherein the keratinous tissue is selected from hair or nails.

Embodiment 33 is the method of any one of Embodiments 19 to 32, wherein administering to the tissue a compound of Formula II comprises administering to the tissue a composition comprising a compound of Formula II, and wherein the composition further comprises a vehicle.

Embodiment 34 is the method of claim 33, wherein the vehicle is selected from a medium chain triglyceride (MCT), a semifluorinated alkane, or combinations thereof.

Embodiment 35 is the method of claim 34, wherein the MCT is a triglyceride of fatty acids, and the fatty acids are selected from the group consisting of hexanoic acid, octanoic acid, decanoic acid, dodecanoic acid, and combinations thereof.

Embodiment 36 is the method of any one of Embodiments 34 or 35, wherein the semifluorinated alkane compound has a formula of RFRH or a formula of RFRHRF; RF is a perfluorinated hydrocarbon with 1 to 15 carbon atoms, and wherein RH is a non-fluorinated hydrocarbon with 1 to 15 carbon atoms.

Embodiment 37 is the method of any one of Embodiments 34 to 36, wherein the semifluorinated alkane is selected from perfluorobutylbutane (F4H4), perfluorobutylpentane (F4H5), perfluorobutylhexane (F4H6), perfluorohexylethane (F6H2), perfluorohexylbutane (F6H4), perfluorohexyloctane (F6H8), and perfluorohexyldecane (F6H10); preferably, perfluorohexylhexane (F6H6), and perfluorohexyloctane (F6H8).

Embodiment 38 is the composition of any one of Embodiments 34 to 37, wherein the semifluorinated alkane is perfluorohexyloctane (F6H8).

Embodiment 39 is the method of any one of Embodiments 33 to 38, wherein the compound of Formula II is present in an amount of from about 0.001% to about 1% (w/w), from about 0.01% to about 1% (w/w), from about 0.05% to about 0.5% (w/w), about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), or 1% (w/w).

Embodiment 40 is the method of any one of Embodiments 34 to 39, wherein the MCT is present in an amount of from about 0% to about 99.9% (w/w), from about 0.1% to about 99.9% (w/w), from about 0.1% to about 30% (w/w), from about 1% to about 25% (w/w), from about 1% to about 20% (w/w), from about 5% to about 20% (w/w), from about 5% to about 15% (w/w), from about 10% to about 99.9% (w/w), from about 20% to about 99.9% (w/w), from about 30% to about 99.9% (w/w), from about 40% to about 99.9% (w/w), from about 50% to about 99.9% (w/w), from about 60% to about 99.9% (w/w), from about 65% to about 99.9% (w/w), from about 70% to about 99.9% (w/w), from about 75% to about 99.9% (w/w), from about 80% to about 99.9% (w/w), from about 85% to about 99.9% (w/w), from about 90% to about 99.9% (w/w), from about 95% to about 99.9% (w/w), from about 98% to about 99.9% (w/w), from about 20% to about 80% (w/w), from about 20% to about 70% (w/w), from about 20% to about 60% (w/w), from about 20% to about 50% (w/w), from about 20% to about 40% (w/w), from about 20% to about 30% (w/w), from about 30% to about 80% (w/w), from about 40% to about 80% (w/w), from about 50% to about 80% (w/w), from about 60% to about 80% (w/w), from about 70% to about 80% (w/w), about 99.9% (w/w), about 99% (w/w), about 98% (w/w), about 97% (w/w), about 96% (w/w), about 95% (w/w), about 94% (w/w), about 93% (w/w), about 92% (w/w), about 91% (w/w), about 90% (w/w), about 85% (w/w), about 80% (w/w), about 75% (w/w), about 70% (w/w), about 65% (w/w), about 60% (w/w), about 55% (w/w), about 50% (w/w), about 45% (w/w), about 40% (w/w), about 35% (w/w), about 30% (w/w), about 25% (w/w), about 20% (w/w), about 15% (w/w), about 10% (w/w), about 9% (w/w), about 8% (w/w), about 7% (w/w), about 6% (w/w), about 5% (w/w), about 4% (w/w), about 3% (w/w), about 2% (w/w), about 1% (w/w), about 0.5% (w/w), or about 0.1% (w/w).

Embodiment 41 is the method of any one of Embodiments 34 to 40, wherein the semifluorinated alkane is present in an amount of from about 0% to about 99.9% (w/w), from about 0.1% to about 99.9% (w/w), from about 0.1% to about 30% (w/w), from about 1% to about 25% (w/w), from about 1% to about 20% (w/w), from about 5% to about 20% (w/w), from about 5% to about 15% (w/w), from about 10% to about 99.9% (w/w), from about 20% to about 99.9% (w/w), from about 30% to about 99.9% (w/w), from about 40% to about 99.9% (w/w), from about 50% to about 99.9% (w/w), from about 60% to about 99.9% (w/w), from about 65% to about 99.9% (w/w), from about 70% to about 99.9% (w/w), from about 75% to about 99.9% (w/w), from about 80% to about 99.9% (w/w), from about 85% to about 99.9% (w/w), from about 90% to about 99.9% (w/w), from about 95% to about 99.9% (w/w), from about 98% to about 99.9% (w/w), from about 20% to about 80% (w/w), from about 20% to about 70% (w/w), from about 20% to about 60% (w/w), from about 20% to about 50% (w/w), from about 20% to about 40% (w/w), from about 20% to about 30% (w/w), from about 30% to about 80% (w/w), from about 40% to about 80% (w/w), from about 50% to about 80% (w/w), from about 60% to about 80% (w/w), from about 70% to about 80% (w/w), about 99.9% (w/w), about 99% (w/w), about 98% (w/w), about 97% (w/w), about 96% (w/w), about 95% (w/w), about 94% (w/w), about 93% (w/w), about 92% (w/w), about 91% (w/w), about 90% (w/w), about 85% (w/w), about 80% (w/w), about 75% (w/w), about 70% (w/w), about 65% (w/w), about 60% (w/w), about 55% (w/w), about 50% (w/w), about 45% (w/w), about 40% (w/w), about 35% (w/w), about 30% (w/w), about 25% (w/w), about 20% (w/w), about 15% (w/w), about 10% (w/w), about 9% (w/w), about 8% (w/w), about 7% (w/w), about 6% (w/w), about 5% (w/w), about 4% (w/w), about 3% (w/w), about 2% (w/w), about 1% (w/w), about 0.5% (w/w), or about 0.1% (w/w).

Embodiment 42 is the method of any one of Embodiments 34 to 41, wherein the MCT is present in an amount of about 1% (w/w) and the SFA is present in an amount of about 99% (w/w), wherein the MCT is present in an amount of about 2% (w/w) and the SFA is present in an amount of about 98% (w/w), wherein the MCT is present in an amount of about 5% (w/w) and the SFA is present in an amount of about 95% (w/w), wherein the MCT is present in an amount of about 10% (w/w) and the SFA is present in an amount of about 90% (w/w), wherein the MCT is present in an amount of about 15% (w/w) and the SFA is present in an amount of about 85% (w/w), wherein the MCT is present in an amount of about 20% (w/w) and the SFA is present in an amount of about 80% (w/w), wherein the MCT is present in an amount of about 25% (w/w) and the SFA is present in an amount of about 75% (w/w), wherein the MCT is present in an amount of about 30% (w/w) and the SFA is present in an amount of about 70% (w/w), wherein the MCT is present in an amount of about 35% (w/w) and the SFA is present in an amount of about 65% (w/w), wherein the MCT is present in an amount of about 40% (w/w) and the SFA is present in an amount of about 60% (w/w), wherein the MCT is present in an amount of about 45% (w/w) and the SFA is present in an amount of about 55% (w/w), wherein the MCT is present in an amount of about 50% (w/w) and the SFA is present in an amount of about 50% (w/w), wherein the MCT is present in an amount of about 55% (w/w) and the SFA is present in an amount of about 45% (w/w), wherein the MCT is present in an amount of about 60% (w/w) and the SFA is present in an amount of about 40% (w/w), wherein the MCT is present in an amount of about 65% (w/w) and the SFA is present in an amount of about 35% (w/w), wherein the MCT is present in an amount of about 70% (w/w) and the SFA is present in an amount of about 30% (w/w), wherein the MCT is present in an amount of about 75% (w/w) and the SFA is present in an amount of about 25% (w/w), wherein the MCT is present in an amount of about 80% (w/w) and the SFA is present in an amount of about 20% (w/w), wherein the MCT is present in an amount of about 85% (w/w) and the SFA is present in an amount of about 15% (w/w), wherein the MCT is present in an amount of about 90% (w/w) and the SFA is present in an amount of about 10% (w/w), wherein the MCT is present in an amount of about 95% (w/w) and the SFA is present in an amount of about 5% (w/w), wherein the MCT is present in an amount of about 98% (w/w) and the SFA is present in an amount of about 2% (w/w), wherein the MCT is present in an amount of about 99% (w/w) and the SFA is present in an amount of about 1% (w/w), wherein MCT is the only vehicle and the MCT is present in an amount of about 99% (w/w), wherein MCT is the only vehicle and the MCT is present in an amount of about 99.5% (w/w), wherein MCT is the only vehicle and the MCT is present in an amount of about 99.6% (w/w), wherein MCT is the only vehicle and the MCT is present in an amount of about 99.7% (w/w), wherein MCT is the only vehicle and the MCT is present in an amount of about 99.8% (w/w), wherein MCT is the only vehicle and the MCT is present in an amount of about 99.9% (w/w), wherein SFA is the only vehicle and the SFA is present in an amount of about 99% (w/w), wherein SFA is the only vehicle and the SFA is present in an amount of about 99.5% (w/w), wherein SFA is the only vehicle and the SFA is present in an amount of about 99.6% (w/w), wherein SFA is the only vehicle and the SFA is present in an amount of about 99.7% (w/w), wherein SFA is the only vehicle and the SFA is present in an amount of about 99.8% (w/w), or wherein SFA is the only vehicle and the SFA is present in an amount of about 99.9% (w/w).

Embodiment 43 is the method of any one of Embodiments 33 to 42, wherein the composition consists essentially of the compound of Formula II and the vehicle.

Embodiment 44 is the method of any one of Embodiments 33 to 43, wherein the composition is an organic composition without aqueous components.

Embodiment 45 is the method of any one of Embodiments 43 to 44, wherein the composition is a topical composition or a topical ocular composition, and wherein the composition is a solution, a suspension, or an emulsion.

Embodiment 46 is the method of any one of Embodiments 19 to 31 or 33 to 45, wherein the tissue is corneal tissue.

Embodiment 47 is the method of claim 46, wherein the subject has a chemical burn or a disease selected from keratoconus, thin corneas, keratitis, infectious keratitis including bacterial or fungal keratitis, corneal ulcer, bullous keratopathy and other forms of corneal edema, keratolysis, an autoimmune disease, a cicatrizing disease such as Ocular Cicatricial Pemphigoid, Stevens-Johnson Syndrome, Lupus Erythematosus, Rheumatoid Arthritis, corneal ectasia including cornea ectasia appearing after laser-assisted in situ keratomileusis (LASIK) and photorefractive keratectomy (PRK), post-refractive ectasia, corneal degeneration, brittle cornea syndrome, Ehlers-Danlos Syndrome Type VI, corneal neovascularization, corneal melting including Corneal Melting in Boston Keratoprosthesis Type I, hyperopia, hyperopic astigmatism, myopia, astigmatism, myopic astigmatism, myopic regression, keratopathy, corneal astigmatism, unstable cornea, or pellucid marginal degeneration (PMD).

Embodiment 48 is the method of any one of Embodiments 46 to 47, wherein the composition is in the form of an eye drop and the method further comprises applying the eye drop to an eye of the subject.

Embodiment 49 is the method of any one of Embodiments 46 to 48, further comprising de-epithelializing at least a portion of the cornea.

Embodiment 50 is the method of any one of Embodiments 46 to 48, wherein the crosslinking is performed without de-epithelializing any portion of the cornea.

Embodiment 51 is a method for crosslinking collagen in a tissue of a subject, comprising:
administering to the tissue of the subject the composition of any one of Embodiments 1 to 18; and
irradiating the tissue of the subject with UV radiation or blue light.

Embodiment 52 is a method for crosslinking a structural protein, comprising:
contacting the structural protein or a biological tissue containing the structural protein with the composition of any one of Embodiments 1 to 18; and
irradiating the structural protein or the tissue of the subject with UV radiation or blue light.

Embodiment 53 is the method of any one of Embodiments 51 to 52, wherein the structural protein is present in a biological tissue or is present in or on a body of a subject.

Embodiment 54 is the method of any one of Embodiments 51 to 53, wherein the biological tissue is ex vivo.

Embodiment 55 is the method of any one of Embodiments 51 to 53, wherein the biological tissue is in or on a body of a subject.

Embodiment 56 is the method of any one of Embodiments 51 to 55, wherein contacting the structural protein or contacting the tissue comprises administering the composition to a tissue in a body of the subject.

Embodiment 57 is the method of any one of Embodiments 51 to 56, wherein the tissue is selected from a collagenous tissue, epidermal tissue, or keratinous tissue.

Embodiment 58 is the method of claim 57, wherein the keratinous tissue is selected from hair or nails.

Embodiment 59 is the method of any one of Embodiments 51 to 56, wherein the tissue is corneal tissue.

Embodiment 60 is the method of claim 59, wherein the subject has a chemical burn or a disease selected from keratoconus, thin corneas, keratitis, infectious keratitis including bacterial or fungal keratitis, corneal ulcer, bullous keratopathy and other forms of corneal edema, keratolysis, an autoimmune disease, a cicatrizing disease such as Ocular Cicatricial Pemphigoid, Stevens Johnson Syndrome, Lupus Erythematosus, Rheumatoid Arthritis, corneal ectasia including cornea ectasia appearing after laser-assisted in situ keratomileusis (LASIK) and photorefractive keratectomy (PRK), post-refractive ectasia, corneal degeneration, brittle cornea syndrome, Ehlers-Danlos Syndrome Type VI, corneal neovascularization, corneal melting including Corneal Melting in Boston Keratoprosthesis Type I, hyperopia, hyperopic astigmatism, myopia, astigmatism, myopic astigmatism, myopic regression, keratopathy, corneal astigmatism, unstable cornea, or pellucid marginal degeneration (PMD).

Embodiment 61 is the method of any one of Embodiments 59 to 60, wherein the composition is in the form of an eye drop and the method further comprises applying the eye drop to an eye of the subject.

Embodiment 62 is the method of any one of Embodiments 59 to 61, further comprising de-epithelializing at least a portion of the cornea.

Embodiment 63 is the method of any one of Embodiments 59 to 61, wherein the crosslinking is performed without de-epithelializing any portion of the cornea.

Embodiment 64 is a method for crosslinking collagen in a cornea of a subject, comprising:
administering to at least a portion of the cornea of the subject the composition of any one of Embodiments 1 to 18; and
irradiating at least a portion of the cornea of the subject with UV radiation or blue light.

Embodiment 65 is the method of claim 64, wherein the subject has a chemical burn or a disease selected from keratoconus, thin corneas, keratitis, infectious keratitis including bacterial or fungal keratitis, corneal ulcer, bullous keratopathy and other forms of corneal edema, keratolysis, an autoimmune disease, a cicatrizing disease such as Ocular Cicatricial Pemphigoid, Stevens-Johnson Syndrome, Lupus Erythematosus, Rheumatoid Arthritis, corneal ectasia including cornea ectasia appearing after laser-assisted in situ keratomileusis (LASIK) and photorefractive keratectomy (PRK), post-refractive ectasia, corneal degeneration, brittle cornea syndrome, Ehlers-Danlos Syndrome Type VI, corneal neovascularization, corneal melting including Corneal Melting in Boston Keratoprosthesis Type I, hyperopia, hyperopic astigmatism, myopia, astigmatism, myopic astigmatism, myopic regression, keratopathy, corneal astigmatism, unstable cornea, or pellucid marginal degeneration (PMD).

Embodiment 66 is the method of any one of Embodiments 64 to 65, wherein the composition is in the form of an eye drop and the method further comprises applying the eye drop to an eye of the subject.

Embodiment 67 is the method of any one of Embodiments 64 to 66, further comprising de-epithelializing at least a portion of the cornea.

Embodiment 68 is the method of any one of Embodiments 64 to 66, wherein the crosslinking is performed without de-epithelializing any portion of the cornea.

Embodiment 69 is the composition of any one of Embodiments 1 to 18, or the method of any one of Embodiments 33 to 63 or 64 to 69, wherein the composition is preservative-free or free of ocular irritants.

1. A composition comprising:
a compound of Formula I: 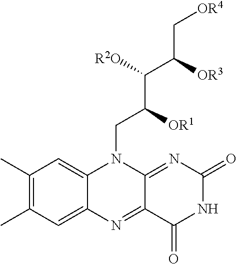
wherein:
R1, R2, and R3 are each independently selected from hydrogen or —C(O)R⁵;
R4 is selected from hydrogen, —C(O)R⁵, a phosphate, or a salt thereof; and
R5 is a linear or branched or cyclic alkyl having 1 to 26 carbon atoms; and
a vehicle selected from a medium chain triglyceride (MCT), a semifluorinated alkane, or combinations thereof.

2. The composition of claim 1, wherein the MCT is a triglyceride of fatty acids, and the fatty acids are selected from the group consisting of hexanoic acid, octanoic acid, decanoic acid, dodecanoic acid, and combinations thereof.

3. The composition of claim 1, wherein the semifluorinated alkane is selected from perfluorobutylbutane (F4H4), perfluorobutylpentane (F4H5), perfluorobutylhexane (F4H6), perfluorohexylethane (F6H2), perfluorohexylbutane (F6H4), perfluorohexyloctane (F6H8), and perfluorohexyldecane (F6H10); preferably, perfluorohexylhexane (F6H6), and perfluorohexyloctane (F6H8).

4. The composition of claim 1, wherein the compound of Formula I is present in an amount of from about 0.001% to about 1% (w/w), the MCT is present in an amount of from about 0.1% to about 99.9% (w/w), and the semifluorinated alkane is present in an amount of from about 0.1% to about 99.9% (w/w).

5. The composition of claim 1, wherein:
R1, R2, R3, and R4 are each independently selected from hydrogen or —C(O)R⁵;
R5 is a linear or branched or cyclic alkyl having 1 to 26 carbon atoms; and
at least one of R1, R2, R3, and R4 is —C(O)R⁵, at least two of R1, R2, R3, and R4 is —C(O)R⁵, or at least three of R1, R2, R3, and R4 is —C(O)R⁵.

6. The composition of claim 1, wherein the compound of Formula I is selected from riboflavin tetraacetate, riboflavin tetrapropionate, riboflavin tetrabutyrate, riboflavin tetrapalmitate, or combinations thereof.

7. The composition of claim 1, wherein the composition consists essentially of the compound of Formula I and the vehicle.

8. The composition of claim 1, wherein the composition is an organic composition without aqueous components.

9. A method for crosslinking collagen in a tissue of a subject, comprising:
administering to the tissue of the subject the composition of claim 1, or administering to the tissue of the subject a compound of Formula II: 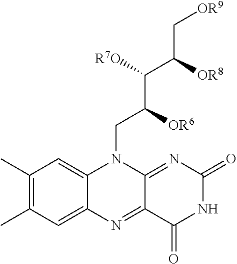
wherein:
R6, R7, R8, and R9 are each independently selected from hydrogen or —C(O)R5;
at least one of R6, R7, R8, and R9 is —C(O)R⁵; and
R5 is selected from a linear or branched or cyclic alkyl having 1 to 26 carbon atoms; and
irradiating the tissue with UV radiation or blue light.

10. A method for crosslinking a structural protein, comprising:
contacting the structural protein or a biological tissue containing the structural protein with the composition of claim 1, or contacting the structural protein or a biological tissue containing the structural protein with a compound of Formula II: 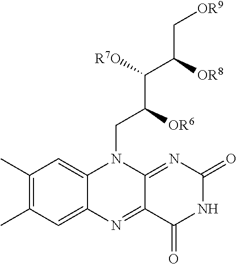
wherein:
R6, R7, R8, and R9 are each independently selected from hydrogen or —C(O)R⁵;
at least one of R6, R7, R8, and R9 is —C(O)R⁵; and
R5 is selected from a linear or branched or cyclic alkyl having 1 to 26 carbon atoms; and
irradiating the structural protein or the tissue with UV radiation or blue light.

11. The method of claim 10, wherein the structural protein is selected from collagen, keratin, derivatives thereof, and combinations thereof.

12. The method of claim 9, wherein the tissue is selected from a collagenous tissue, epidermal tissue, or keratinous tissue.

13. The method of claim 9, wherein the tissue is corneal tissue.

14. The method of claim 13, wherein the subject has a chemical burn or a disease selected from keratoconus, thin corneas, keratitis, infectious keratitis including bacterial or fungal keratitis, corneal ulcer, bullous keratopathy and other forms of corneal edema, keratolysis, an autoimmune disease, a cicatrizing disease such as Ocular Cicatricial Pemphigoid, Stevens Johnson Syndrome, Lupus Erythematosus, Rheumatoid Arthritis, corneal ectasia including cornea ectasia appearing after laser-assisted in situ keratomileusis (LASIK) and photorefractive keratectomy (PRK), post-refractive ectasia, corneal degeneration, brittle cornea syndrome, Ehlers-Danlos Syndrome Type VI, corneal neovascularization, corneal melting including Corneal Melting in Boston Keratoprosthesis Type I, hyperopia, hyperopic astigmatism, myopia, astigmatism, myopic astigmatism, myopic regression, keratopathy, corneal astigmatism, unstable cornea, or pellucid marginal degeneration (PMD).

15. The method of claim 13, further comprising de-epithelializing at least a portion of the cornea.

16. The method of claim 13, wherein the crosslinking is performed without de-epithelializing any portion of the cornea.

17. A method for crosslinking collagen in a cornea of a subject, comprising:
administering to at least a portion of the cornea of the subject the composition of claim 1; and
irradiating at least a portion of the cornea of the subject with UV radiation or blue light.

18. The method of claim 17, wherein the subject has a chemical burn or a disease selected from keratoconus, thin corneas, keratitis, infectious keratitis including bacterial or fungal keratitis, corneal ulcer, bullous keratopathy and other forms of corneal edema, keratolysis, an autoimmune disease, a cicatrizing disease such as Ocular Cicatricial Pemphigoid, Stevens Johnson Syndrome, Lupus Erythematosus, Rheumatoid Arthritis, corneal ectasia including cornea ectasia appearing after laser-assisted in situ keratomileusis (LASIK) and photorefractive keratectomy (PRK), post-refractive ectasia, corneal degeneration, brittle cornea syndrome, Ehlers-Danlos Syndrome Type VI, corneal neovascularization, corneal melting including Corneal Melting in Boston Keratoprosthesis Type I, hyperopia, hyperopic astigmatism, myopia, astigmatism, myopic astigmatism, myopic regression, keratopathy, corneal astigmatism, unstable cornea, or pellucid marginal degeneration (PMD).

19. The method of claim 17, further comprising de-epithelializing at least a portion of the cornea.

20. The method of claim 17, wherein the crosslinking is performed without de-epithelializing any portion of the cornea.
